(19) United States
(12) Patent Application Publication
(10) Pub. No.: US 2019/0282307 A1
(43) Pub. Date: Sep. 19, 2019

(54) DUAL-MODE IMAGING SYSTEM FOR TRACKING AND CONTROL DURING MEDICAL PROCEDURES

(71) Applicant: Children's National Medical Center, Washington, DC (US)

(72) Inventors: Mahdi AZIZIAN, Sunnyvale, CA (US); Peter Kim, Washington, DC (US); Axel Krieger, Alexandria, VA (US); Simon Leonard, State College, PA (US); Azad Shademan, Campbell, CA (US); Ryan Decker, Pittsburgh, PA (US); Justin Opfermann, Washington, DC (US); Matthieu Dumont, Tallahassee, FL (US); Nick Uebele, Colleyville, TX (US); Lydia Carroll, Bethesda, MD (US); Ryan Walter, Baltimore, MD (US); Rohan Fernandes, Bethesda, MD (US)

(73) Assignee: Children's National Medical Center, Washington, DC (US)

(21) Appl. No.: 16/364,067

(22) Filed: Mar. 25, 2019

Related U.S. Application Data
(63) Continuation-in-part of application No. 13/863,954, filed on Apr. 16, 2013.
(60) Provisional application No. 62/647,141, filed on Mar. 23, 2018.
(60) Provisional application No. 61/624,665, filed on Apr. 16, 2012.

(51) Int. Cl. A61B 34/20; A61B 90/00

(52) U.S. Cl. CPC A61B 34/20; A61B 90/361; A61B 2090/3983; A61B 2090/3937; A61B 2034/2055

(57) ABSTRACT

System and method for tracking and control in medical procedures. The system including a device that deploys fluorescent material on at least one of an organ under surgery and a surgical tool, a visual light source, a fluorescent light source corresponding to an excitation wavelength of the fluorescent material, an image acquisition and control element that controls the visual light source and the fluorescent light source, and captures and digitizes at least one of resulting visual images and fluorescent images, and an image-based, tracking module that applies image processing to the visual and fluorescent images, the image processing detecting fluorescent markers on at least one of the organ and the surgical tool.

### CROSS-REFERENCE TO RELATED APPLICATIONS

The present application claims priority to U.S. Provisional Application No. 62/647,141, filed on Mar. 23, 2018, the teaching of which is hereby incorporated by reference in its entirety for all purposes.

The present application further claims priority to U.S. application Ser. No. 13/863,954, filed on Apr. 16, 2013, which claims priority U.S. Provisional Application No. 61/1624,665, filed on Apr. 16, 2012, the teachings of which are hereby incorporated by reference in their entirety for all purposes.

### BACKGROUND OF THE INVENTION

### Field of the Invention

The present embodiments relate generally to apparatuses and methods for tracking and control in surgery and interventional medical procedures.

### Description of the Related Art

There is currently no technology for robust image-guidance in automated surgery. What is available in the market as so called “robotic surgery” is truly just robot-assisted surgery because the robot only follows direct commands of the surgeon with very little intelligence or autonomy. Some research groups have looked into closing the loop of control for surgical robots with existing sensors, however, special conditions and considerations applied to operations in vivo make it extremely difficult to achieve such goals.

### SUMMARY OF THE INVENTION

The present embodiments address at least this problem by introducing a robust tracking technique which requires minimal changes to the current robot-assisted surgical workflow and closing the loop with an effector function.

The foregoing paragraphs have been provided by way of general introduction, and are not intended to limit the scope of the following claims. The described embodiments, together with further advantages, will be best understood, by reference to the following detailed, description taken in conjunction with the accompanying drawings.

### BRIEF DESCRIPTION OF THE DRAWINGS

A more complete appreciation of the embodiments described herein, and many of the attendant advantages thereof will be readily obtained as the same becomes better understood by reference to the following detailed description when considered in connection with the accompanying drawings, wherein

FIG. 1 shows the overall structure of the invention in semi-autonomous mode where the surgical tasks are partially automated by visual servoing, according to an exemplary embodiment of the present disclosure;

FIG. 2 shows the system in the manual or master-slave robot-assisted mode, according to an exemplary embodiment of the present disclosure;

FIG. 3 represents the system with supervised autonomy, according to an exemplary embodiment of the present disclosure;

FIG. 4 shows a spectral range of the excitation and emission lights which describes the distinct spectral ranges associated with the main components involved (i.e., hemoglobin's (oxygenated and deoxygenated), water and the fluorescent dye), wherein fluorescent dyes with different spectral ranges for excitation and emission can be synthesized (e.g. Cyanine dyes), according to an exemplary embodiment of the present disclosure;

FIG. 5 illustrates an example of markers placed around a phantom cut, according to an exemplary embodiment of the present disclosure;

FIG. 6 illustrates images captured using a near infrared camera, with two example fluorescent agents, according to an exemplary embodiment of the present disclosure;

FIG. 7 illustrates stereo image formation and triangulation to extract three dimensional (3D) coordinates of NIR marker, according to an exemplary embodiment of the present disclosure;

FIG. 8 illustrates a flow diagram for an exemplary robotic operation algorithm, according to an exemplary embodiment of the present disclosure;

FIG. 9 illustrates a flow diagram for another exemplary robotic operation algorithm, according to an exemplary embodiment of the present disclosure;

FIG. 10 illustrates a flow diagram for a method, according to an exemplary embodiment of the present disclosure;

FIG. 11 illustrates a block diagram of a computing , according to an exemplary embodiment of the present disclosure

FIG. 12 describes composition of the agents and possible agents used to create a surgical marker, wherein the marker includes visibility, binding, and stability agents that are mixed together in a ratio dependent on the agents used, according to an exemplary embodiment of the present disclosure;

FIG. 13A is an illustration of a solid marker using mechanical force as a bind mechanism between a marker and a target tissue, according to an exemplary embodiment of the present disclosure;

FIG. 13B is an illustration of a solid -Harker using a piercing mechanism to mechanically hold a marker in place relative to a tissue, according to an exemplary embodiment of the present disclosure;

FIG. 14A is an image of a circular marker applied to target tissue via syringe and needle, according to an exemplary embodiment of the present disclosure;

FIG. 14B is an image of a syringe and needle with a dispensing, tip having a shape such that the liquid marker is circular on the target tissue, according to an exemplary embodiment of the present disclosure;

FIG. 15A is an image of a liquid marker applied to a target tissue with a marking pen and felt dispensing nib, according to an exemplary embodiment of the present disclosure;

FIG. 15B is an image of a marking pen and felt dispensing nib, the shape of the dispensing nib being such that the liquid marker is applied in a linear pattern on the target tissue, according to an exemplary embodiment of the present disclosure;

FIG. 16 is an illustration of a speckle pattern created by spraying a liquid marker onto target tissue, wherein the speckled pattern may be used to differentiate between the background and foreground of the surgical field, according to an exemplary embodiment of the present disclosure;

FIG. 17 is an illustration of an implementation of a mask used to create a specific marking pattern during spraying of a liquid marker onto a target tissue, according to an exemplary embodiment of the present disclosure;

FIG. 18A is an illustration of a solid marker in a checkerboard pattern that can be applied to a tool in the surgical field for tracking and positioning of the tool to automatically carry out a surgical task, according to an exemplary embodiment of the present disclosure;

FIG. 18B is an illustration of a solid marker in a checkerboard pattern that can be applied to a tool in the surgical field for tracking and positioning of the tool to automatically carry out a surgical task, according to an exemplary embodiment of the present disclosure;

FIG. 19 is an image of near-infrared fluorescent silicone molded into a ball shape and exposed to near-infrared light, according to an exemplary embodiment of the present disclosure;

FIG. 20 is an image of a marking apparatus for applying a liquid marker to a target tissue, the liquid marker being stored in a reservoir and dispensed through a tip when the pump is activated and wherein fine adjustments can be made to the pump to tightly control the amount of marker dispensed, according to an exemplary embodiment of the present disclosure;

FIG. 21 is an image of a disposable marking apparatus for liquid markers and one or more different tips that can be fixed to the disposable marking apparatus according to a desired marker pattern, according to an exemplary embodiment of the present disclosure;

FIG. 22 is an image of a liquid marking tool including a reservoir, a liquid marker, and a dispensing tip, wherein the reservoir is made from a compliant material that can be squeezed to force the liquid marker through the dispensing tip, according to an exemplary embodiment of the present disclosure;

FIG. 23A is an image of a laparoscopic hand tool for applying liquid markers during laparoscopic surgery, wherein liquid marker capsules can be loaded into the hand tool and applied to target tissue, an application of compressive force to the handles of the laparoscopic tool pushing the liquid marker through the dispensing tip, according to an exemplary embodiment of the present disclosure;

FIG. 23B is an image of a liquid marker capsule that can be loaded into the hand tool and applied to target tissue, according to an exemplary embodiment of the present disclosure;

FIG. 24 is an illustration of a spray gun for application of liquid marker to a target tissue, the spray gun including an extended nozzle for laparoscopic applications, according to an exemplary embodiment of the present disclosure;

FIG. 25 is an illustration of an apparatus for delivering markers to targets, the apparatus having a reservoir for holding liquid marker to be dispensed through a dispensing nozzle, according to an exemplary embodiment of the present disclosure;

FIG. 26 is an illustration of a modified tattoo gun for injecting liquid marker into target tissue, the liquid marker being stored in a reservoir at a dispensing tip, according to an exemplary embodiment of the present disclosure;

FIG. 27 is a near infrared image of a fluorescent suture, passed through both walls of vaginal cuff tissue, used in vivo in a porcine model to stage a vaginal cuff, the corners of the vaginal cuff being marked with a liquid near infrared marker that polymerizes into a solid on contact with tissue, according to an exemplary embodiment of the present disclosure;

FIG. 28A is a fluorescent image used to stage vaginal cuff tissue in an animal model, according to exemplary embodiment of the present disclosure;

FIG. 28B is a segmented image used to determine suture position for robot positioning and control, wherein dots on the periphery indicate the center locations of the sutures, a centrally-located dot indicates the center of the target tissue, location of the vaginal tissue, and the positional information can be used to, detect stay suture locations, according to an exemplary embodiment of the present disclosure;

FIG. 29 illustrates a marking apparatus designed to keep each agent separated until the appropriate mixing time, according to an exemplary embodiment of the present disclosure;

FIG. 30 is an illustration of a syringe-based mixing system with modifications for mixing and dispensing all agents used in a liquid marker through a dispensing tip, each agent being maintained in separate storage containers prior to injection into a mixing reservoir of the syringe and being mixed by pumping action, according to an exemplary embodiment of the present disclosure;

FIG. 31 is an illustration of a use of different marker shapes in distinguishing a background of an image from a foreground of an image during a surgical procedure, according to an exemplary embodiment of the present disclosure;

FIG. 32 is an illustration of a use of different markers in distinguishing between different tissue types in a surgical setting with similar tissue colors, according to an exemplary embodiment of the present disclosure;

FIG. 33 is an illustration of linear markers used to define no fly regions of a surgical setting, region one being defined by a linear marker border and containing additional markers on the tissue, region two being defined by a completely enclosed linear marker border, and region three being defined by a single linear marker and additional input from a user, according to an exemplary embodiment of the present disclosure; and

FIG. 34 is an image of liquid markers used to identify landmarks on target tissue, the markers indicating a location for an automated robotic platform to complete a surgical task, according to an embodiment of the present disclosure.

### DETAILED DESCRIPTION OF THE PREFERRED EMBODIMENTS

The terms “a” or “an”, as used herein, are defined as one or more than one. The term “plurality”, as used herein, is defined as two or more than two. The term “another”, as used herein, is defined as at least a second or more. The terms “including” and/or “having”, as used herein, are defined as comprising (i.e., open language). Reference throughout this document to “one embodiment”, “certain embodiments”, “an embodiment”, “an implementation”, “an example” or similar terms means that a particular feature, structure, or characteristic described in connection with the embodiment is included in at least one embodiment of the present disclosure. Thus, the appearances of such phrases or in various places throughout this specification are not necessarily all referring to the same embodiment. Furthermore, the particular features, structures, or characteristics may be combined in any suitable manner in one or more embodiments without limitation.

According to an embodiment, the present disclosure describes a system for tracking and control in medical procedures. The system includes a device configured to deploy fluorescent material on at least one of an organ under surgery and a surgical tool, a visual light source, a fluorescent light source corresponding to an excitation wavelength of the fluorescent material, an image acquisition and control element configured to control the visual light source and the fluorescent light source, and configured to capture and digitize at least one of resulting visual images and fluorescent images, and an image-based tracking module configured to apply image processing to the visual and fluorescent images, the image processing detecting fluorescent markers on at least one of the organ and the surgical tool.

According to another embodiment of the present disclosure, the system further includes in the system a surgical robot and a visual servoing control module configured to receive tracking information from the image-based tracking module and to control the surgical robot, based on the tracking information, to perform a surgical operation.

According to another embodiment of the present disclosure, the system further includes a surgical robot and a visual servoing control module configured to receive tracking information from the image-based tracking module and to control the surgical robot, based on the tracking information, to perform a surgical operation.

According to another embodiment of the present disclosure, the system further includes a manual control module configured to enable manual control of the surgical robot in place of control by the visual servoing control module.

According to another embodiment of the present disclosure, the visual servoing control module is further configured to receive manual input and to control the surgical robot, based on the manual input, to perform a surgical operation.

According to another embodiment of the present disclosure, the system further includes a surgical robot and a manual control module configured to receive manual input and execute master-slave control of the surgical robot.

According to another embodiment of the present disclosure, the system further includes a display configured to display at least one of the visual images and the fluorescent images.

According to another embodiment of the present disclosure, the image-based tracking module further identifies the organ or the surgical tool based on the detected fluorescent markers.

According to another embodiment of the present disclosure, the image acquisition and control element further includes a dynamic tunable filter configured to alternatively pass visual light and light emitted by the fluorescent material, and a charged coupled device configured to capture at least one of visual images and fluorescent images.

According to another embodiment of the present disclosure, the display is stereoscopic or monoscopic.

According to another embodiment of the present disclosure, the image acquisition and control element generates stereoscopic or monoscopic images.

According to another embodiment of the present dislcosure, the stereoscopic display is further configured to display visual images and a color coded overlay of fluorescent images.

According to another embodiment of the present disclosure, the stereoscopic display is further configured to display an augmented reality image by overlaying target points detected by the image-based tracking module.

According to another embodiment of the present disclosure, the system is configured to provide at least one of visual, audio, and haptic feedback to a system operator, based on information provided by the image-based tracking module.

According to another embodiment of the present disclosure, the system is configured to operate in each of a manual mode, a semi-autonomous mode, and an autonomous mode.

According to another embodiment of the system, the image-based tracking module identifies virtual boundaries based on the detected fluorescent markers to designate critical structures.

According to another embodiment of the present disclosure, the system further includes a detection device configured to determine whether a surgical tool has passed a boundary and to provide constraints on motion or provide alarms when the boundary has been crossed in order to protect the critical structures.

According to another embodiment of the present disclosure, the fluorescent light source is a near-infrared (NIR) light source.

According to another embodiment of the present disclosure, the image acquisition and control element includes two charge coupled devices (CCDs), one assigned to a visual spectrum and one assigned to a NIR spectrum.

According to another embodiment of the present disclosure, light generated by the visual light source and the fluorescent light source is split by either a beam-splitting or a dichromatic prism.

According to another embodiment of the present disclosure, light generated by the visual light source and the fluorescent light source are provided separate light paths to the two CCDs.

According to one embodiment, the present disclosure describes a method for performing a medical procedure. The method includes the steps of deploying fluorescent material on at least one of an organ under surgery and a surgical tool, illuminating the organ, the surgical tool, or both, with a visual light source and a fluorescent light source, the fluorescent light source corresponding to an excitation wavelength of the fluorescent material, capturing and digitizing images resulting from the illumination by the visual light source and the fluorescent light source, and applying image processing to the digitized images, the image processing detecting fluorescent markers on at least one of the organ and the surgical tool.

According to another embodiment, the method of the present disclosure further includes generating tracking information by tracking the organ, the surgical tool, or both based on the detected fluorescent markers.

According to another embodiment, the method of the present disclosure further includes controlling a surgical robot, based on the tracking information, to perform a surgical operation.

According to another embodiment, the method of the present disclosure further includes receiving manual input and controlling the surgical robot, based on the manual input, to perform the surgical operation.

According to another embodiment, the method of the present disclosure further includes receiving manual input and executing master-slave control of a surgical robot based on the on manual input.

According to another embodiment, the method of the present disclosure further includes providing a stereoscopic or monoscopic display of the digitized images.

According to another embodiment, the method of the present disclosure further includes generating stereoscopic or monoscopic images in order to capture and digitize images.

According to another embodiment, the method of the present disclosure further includes displaying visual images and a color coded overlay of fluorescent images.

According to another embodiment, the method of the present disclosure further includes displaying an augmented reality image by overlaying target points detected by the image-based tracking module.

According to another embodiment, the method of the present disclosure further includes providing at least one of visual, audio, or haptic feedback to a system operator, based on the tracking information.

According to another embodiment, the method of the present disclosure further includes identifying the organ or the surgical tool based on the detected fluorescent markers.

According to another embodiment, the method of the present disclosure further includes performing a surgical procedure based on the detected fluorescent markers.

According to another embodiment, the method of the present disclosure further includes designating critical structures by identifying virtual boundaries based on the detected fluorescent markers.

According to another embodiment, the method of the present disclosure further includes determining whether a surgical tool has passed a boundary and providing constraints on motion or providing alarms when the boundary has been crossed in order to protect the critical structures.

According to one embodiment, the present disclosure describes a system for tracking and control in medical procedures. The system includes means for deploying fluorescent material on at least one of an organ under surgery and a surgical tool, a visual light source, a fluorescent light source corresponding to an excitation wavelength of the fluorescent material, means for controlling the visual light source and the fluorescent light source, means for capturing and digitizing at least one of resulting visual images and fluorescent images, and means for applying image processing to the visual and fluorescent images, the image processing detecting fluorescent markers on at least one of the organ and the surgical tool.

The embodiments disclosed herein may be applied in the field of automated anastomosis, where tubular structures (vessels, bile ducts, urinary tract, etc.) are coapted, or brought into contact, and sealed. An anastomosis is one of the four major steps in every surgery that includes (1) access through incision, (2) exposure and dissection, (3) resection and removal of pathology, and (4) reconstruction and closure, or anastomosis. An anastomosis is currently achieved by suturing or applying clips or glue to the anastomosis site. The anastomosis procedure, itself, may be performed manually or by using robots through master-slave control. Both of these described techniques can be time consuming and cumbersome in an arena where these parameters are at a premium. The present embodiments make it possible for the surgeon to mark the anastomosis site by applying fluorescent markers via, for instance miniature clips, spray, paint, tapes, and the like, which can be detected and tracked using the dual-spectrum imaging technology. In addition, a robotic system can be controlled through visual servoing using this tracking information, in order to apply sutures, clips, glue, weld and the like at specified positions.

According to embodiments, several other applications include but are not limited to:
Automation of other steps of surgery such as exposure and dissection and resection and removal of pathology,
Automated tumor resection and ablation, wherein a tumor can be painted, using a fluorescent dye and a robotic system can be guided and controlled to resect or ablate the tumor, applicable to, for instance, partial nephrectomy, hepatectomy, and the like,
Assisting in manual or master-slave robotic surgery, wherein the system of the present disclosure can be used as a visual guide to surgeons for manual surgeries and master-slave controlled robotic surgery, allowing critical structures to be marked by the surgeons and therefore, in addition to tools, to be clearly visible to the surgeon through the procedure,
Pre-excisional or incisional biopsy localization of sub-surface or deep nodules or lesions in viscera,
As a reference marker for accurate re-approximation, orientation of tissue, or precise reconstruction of surgical area during open surgery
As a positional marker for motion tracking and memory during endoscopic procedure.

Variants of embodiments of the technology are listed below. For instance, the technology can be used with multiple dyes having excitation/emission wavelengths at different wavelengths. This technology can be applied so that different markers can be used for tracking multiple objects. In an embodiment, fluorescent dyes A and B can be used to mark two sides of a tubular structure prior to automated anastomosis.

In an embodiment, dyes, or markers, can be applied to the targets that are internal as well as targets that are external. The fluorescent dye can be attached to the target by clips, staples, and glue or can be applied by painting or spraying. The dye can also be injected to the tissue to mark specific points or can be injected through blood. The dye can be selected in order to bind with specific types of cells to mark specific structures such, for instance, tumors.

In an embodiment, “no-fly zones” or “virtual fixtures” can be provided to prevent surgical tools from approaching critical structures. For instance, the surgeon can mark a critical structure prior to initiating a task and the marked borders can be tracked using the dual-mode imaging technology. Coordinates can be used to force constraints on the motion of the surgical tools during the automated or semi-automated task. It can also be used to provide alarms in manual tasks, including, among others, visual alarms, audio alarms, or haptic alarms.

In an embodiment, the imaging, system can be monoscopic and provide two-dimensional location of the tracked points which can be used for image-based visual servoing. In another embodiment, the imaging system can be stereoscopic and provide three-dimensional location of the tracked structures, thereby being used for image-based or position-based visual servoing.

Embodiments of the present disclosure can be applied to automated or semi-automated applications. The system of the present disclosure can also provide guidance for manual operations through visual feedback, audio feedback, haptic feedback, and the like.

As introduction, automation of a surgical procedure is a challenging task. As the surgical scene is dynamic, deformable organs may occlude a surgeon's view and variations in illumination can make it difficult to robustly track myriad targets and objects inside the patient's body. Several attempts have been made to develop image-based tracking algorithms for minimally invasive and/or open surgeries, but these attempts rely on special conditions and are not robust and therefore, cannot be practically used to control surgical tools or to automate steps of a surgery.

According to an embodiment, the present disclosure addresses these limitations by using a dual-spectrum imaging device capable of imaging in the visual spectrum as well as in the near-infrared (NIR) spectrum. To this end, a surgeon places fluorescent markers on the locations which should be tracked (e.g., tools and tissue). An excitation light can be generated by the imaging device and cause the fluorophores to emit NIR light which can be detected by the imaging device. Due to limited auto-fluorescence of the tissue compared to the fluorescent dyes, and a lack of other NIR sources in the patient's body, the resulting acquired light has a high signal to noise ratio (SNR). This high SNR makes the tracking algorithm robust and reliable. Moreover, NIR light is able to penetrate tissue at a depth greater than visible light, making it possible to track an object even when occluded by another organ, flipped over, covered by blood, and the like. A combination of visual and NIR images, therefore, can be used to make image-based tracking algorithms even more robust.

According to an embodiment, the present disclosure describes a system for automation of surgical tasks. The system is based on deploying fluorescent markers on the organ under surgery and/or on the surgical tool, allowing for tracking of the fluorescent markers in real-time and controlling the surgical tool via visually servoing.

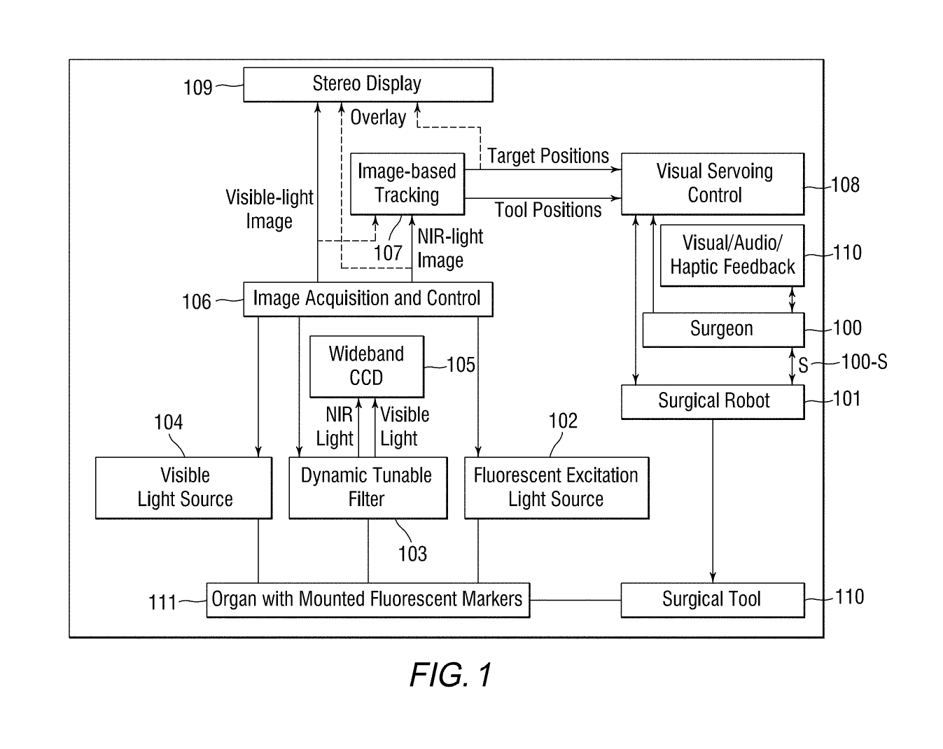

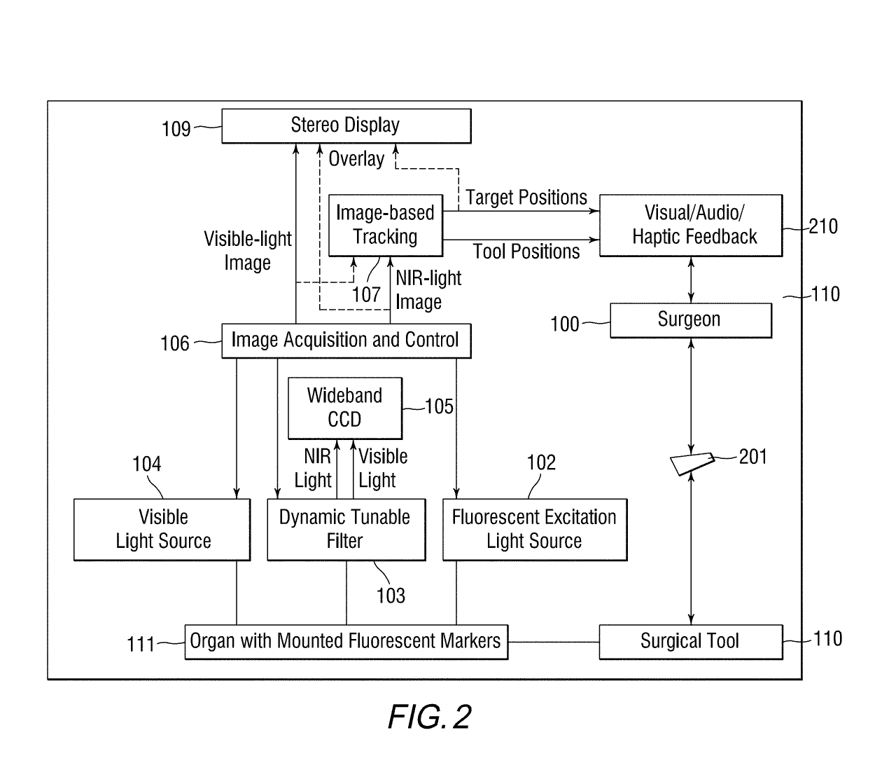

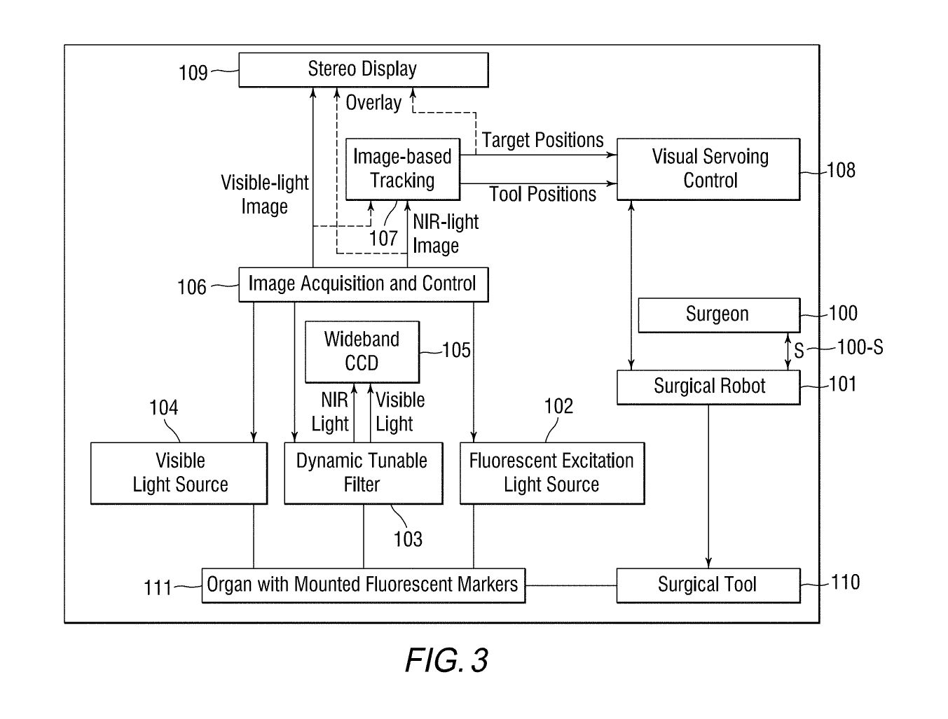

FIG. 1, FIG. 2, and FIG. 3 represent different modes of the operation for the system. Fluorescent markers can be deployed on the organ (e.g. two sides of a bile duct to be anastomosed) through spraying, painting, attachment, or other techniques 111. The markers can also be generated by techniques including mixing of fluorescent dye (e.g., indocyanine green (ICG) mixed with a biocompatible glue, or cyanoacrylate-ICG mix), delivered by pipette, or by spray. The markers can also be generated by any element which provides sufficient fluorescence.

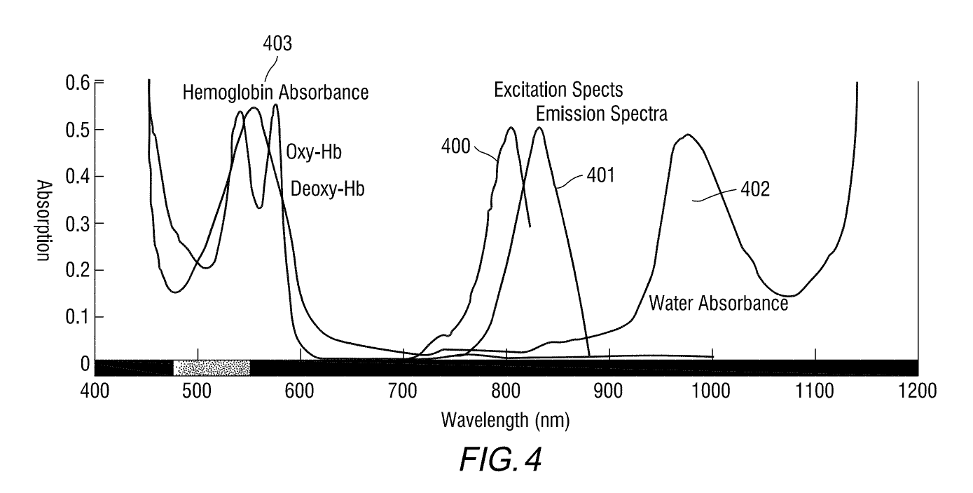

FIG. 4 illustrates spectral characteristics of a fluorescent dye. The separation between excitation wavelength and emission wavelength reduces interference caused by the excitation light source significantly. Fluorescent dye can be chosen to have its emitted wavelength beyond the visible light range in order to achieve a high signal to noise ratio in the near-infrared images. In addition, having the fluorescent emission 400 and excitation 401 wavelengths away from peak absorption wavelengths of water 402 and hemoglobin 403 provides a stronger signal and makes it easier to track fluorescent markers in presence of soft tissue, having a high water content, and blood.
In an embodiment, multiple different markers can be used to help track multiple structures, organs, and tools. Using different markers reduces the error rate for tracking, since the number of similar markers is reduced. Differentiation of markers can be achieved by having different size, different volume, and/or different shape markers and/or using dyes with excitation/emission wavelengths at different wavelengths. In one embodiment, markers with 3 μL volume and markers with 6 μL volume are used to mark the two sides of a tubular structure, respectively, prior to automated anastomosis. In another embodiment, a fluorescent dye emitting at 790 nm corresponds to the “no-fly zone” while a different wavelength 830 nm corresponds to an edge of a structure.
In an embodiment, each structure (e.g., organ, stream segment) is assigned a structure identification number. Likewise, when the surgeon marks a structure at the anastomoses site, each marker is automatically assigned a unique identification number and is automatically labeled with the structure identification number to which it is attached. As the markers are tracked, the label of each marker is used to determine the structure to which it belongs and its overlay color. This tracking may be performed using tables or databases implemented by a computer processor and corresponding software instructions.

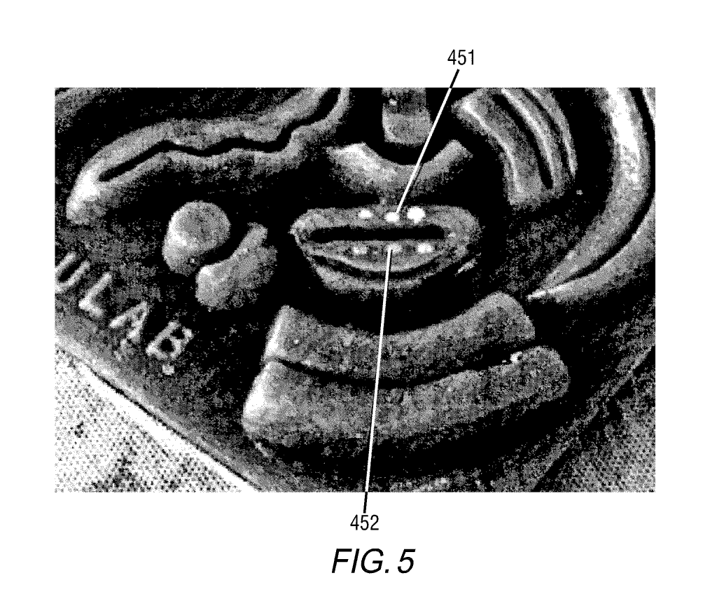

FIG. 5 illustrates markers placed on or around a phantom cut. A first set of markers 451 on the top side of the cut are labeled with a first color (e.g. yellow), and a second set of markers 452 on the bottom side of a cut are labeled with a second color (e.g. green).
FIG. 1 through FIG. 3 illustrate two light sources 102, 104 that illuminate the scene. One light source 104 is a visual light source that makes it possible to acquire normal images of the organs. The other light source 102 is a narrow-band source of light (e.g. in the near infrared range) that is chosen according to the excitation wavelength of the fluorescent material. A “dynamic tunable filter” 103 changes the filter's characteristics in real-time to pass the visual light and the light emitted by the fluorescent material alternatively. At any given moment the filter 103 only passes one type of light while suppressing the other. A wide-band CCD 105 captures images of the received light from either source. The light sources 102, 104, the tunable filter 103, and the image capturing by the CCD 105 are controlled and synchronized by the image acquisition and, control module 106. The image acquisition system runs at a high frame rate (e.g. 60 Hz to 120 Hz) and therefore it acts like two imaging systems with different wavelengths.
According to an embodiment, NIR and visual light can be split by using either a beam-splitting or a dichromatic prism, with two CCDs capturing images, one for the visual spectrum and one for the NIR spectrum. In another embodiment, there may be separate light paths for NIR and visual light and separate CCDs corresponding thereto. These concepts can be simply extended to a multiple wavelength imaging system. In an embodiment, image acquisition and control module 106 captures and digitizes images and provides them to two higher-level modules 107, 109. The stereoscopic display 109 provides the acquired visual images. The stereoscopic display 109t can also display fluorescent images as a color coded overlay or display an augmented reality image by overlaying the target points detected by the image-based tracking module 107. The image-based tracking module 107 can apply image processing algorithms to detect the fluorescent markers in order to track the tools and the organ. Visual features can also be used for tracking.

According to an embodiment, the image-based tracking module 107 also includes, a tracking module that performs pre-processing of the NIR image and visual tracking based on the processed image information. In an embodiment, the pre-processing algorithm can include image processing algorithms, such as image smoothing, to mitigate the effect of sensor noise, image histogram equalization to enhance the pixel intensity values, and image segmentation based on pixel intensity values to extract templates for the NIR markers. The visual trackers can be initialized first. Initialization of the visual trackers starts by detection and segmentation of the NIR marker. Segmentation can be based on applying an adaptive intensity threshold on the enhanced NIR image to obtain a binary template for the NIR markers. A two dimensional (2D) median filter and additional morphology-based binary operators (binary image processing algorithms such as image erosion and dilation) may be applied on the binary template to remove segmentation noise. The binary template may be used as a starting base for visual tracking of NIR markers using visual tracking algorithms. After pre-processing and segmentation, the NIR template can be a white blob on a darker background, the darker background representing the remainder of the surgical field in the NIR image.
In FIG. 1 and FIG. 3, representing “semi-autonomous” and “supervised autonomous” modes, respectively, the surgeon 100 interacts with the surgical robot as a supervisor (100-s), taking over control through a master console whenever required. In the semi-autonomous mode (FIG. 1) the surgeon 100 also provides commands to the visual servoing controller 108 during the operation. The visual servoing controller 108 can receive the tracking information from the image-based tracking module 107, combine these with the intraoperative commands from the surgeon 100, and send appropriate commands to the robot in real-time in order to control the surgical robot 101 and the surgical tool(s) 110 to obtain a predetermined goal (e.g. anastomosis). The surgeon 100 can be provided with visual feedback, audio feedback, or haptic feedback 110 while viewing the stereoscopic display.
In an embodiment, in manual mode (FIG. 2), the surgeon controls the surgical tool manually, as in conventional laparoscopic surgery, or through master-slave control 201 of a robot arm. The surgeon receives visual feedback through the stereoscopic display 109 and may also be provided with other visual feedback, audio feedback, or haptic feedback, but the control loop is solely closed through the surgeon.

In autonomous mode (FIG. 3), the control loop is solely closed via visual servoing except when the surgeon stops the autonomous control and takes over control (100-s) to, among others, prevent a complication or correct for a wrong action.

According to an embodiment, the tracked visual markers can be used to guide the motion of the robot. Each visual marker can be represented by a representative vector of numbers, which is typically called a visual feature. Examples of visual features are coordinates of the centers of NIR markers extracted from the binary image, and/or their higher-order image moments, such as their area in terms of pixels.

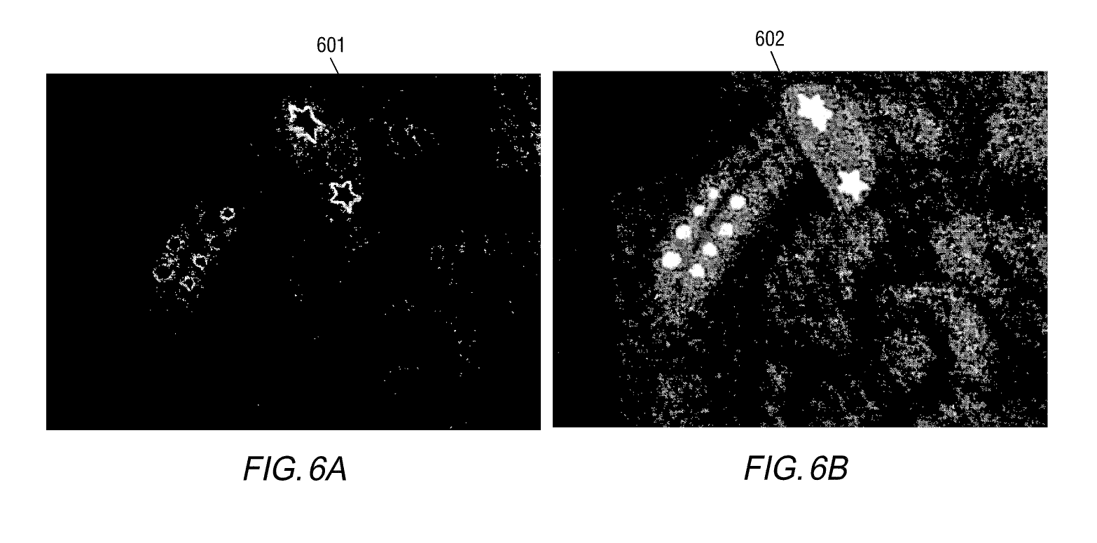

FIG. 6 illustrates images captured using a NIR camera with two exemplary fluorescent agents. Image 601 illustrates a binary image after image processing. Image 602 illustrates data that can be used as visual tracking information.

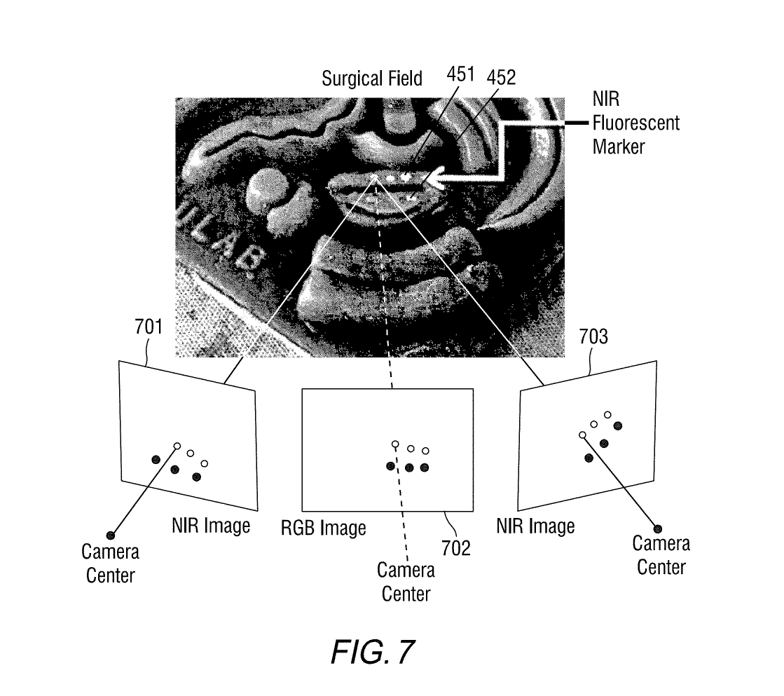

Robotic motion can be performed by transforming the sensor measurements into global Cartesian coordinate form for the robot. In one embodiment, the NIR and the tool markers can be tracked in the stereo images to compute the 3D coordinates of the marker or tool with respect to the surgical field, as shown in FIG. 7. In particular, FIG. 7 illustrates stereo image formation and triangulation to extract three dimensional (3D) coordinates of the NIR markers. These 3D coordinates are used by the robotic motion control algorithm in open loop or closed-loop architecture. The error between the tool position and the marker position is calculated and used to generate the desired tool displacement. When the motion control feedback loop is closed in the sensor space, the effect of calibration errors is limited. This is desired for supervised autonomy. Vision-based and closed, loop feedback motion control of robots is referred to as visual servoing. There are two main approaches to visual servoing based on control architecture: (1) position-based visual servoing (PBVS) and (2) image-based visual servoing (IBVS). In PBVS, the position of the robotic tool is estimated and the error is estimated based on the estimated position and the goal tool position. In IBVS, the image features are used directly to compute the task error in the image space, such that when the robotic tool is at the goal position the task error is zero. Both control approaches generate motions that drive the error to zero.

The NIR based robot motion control is a core technology which has not been developed in the past. Previous methods and apparatuses for NIR based imaging (without robot control. Frangioni 2012, U.S. Pat. No. 8,229.548 B2) and NIR based display (Mohr and Mohr, US 2011/0082369) fail to consider robot motion control or any control whatsoever. With a stereo imaging system consisting of two NIR cameras with appropriate filters, a properly excited NIR agent can be seen in both stereo images. Image processing and visual tracking algorithms, such as the algorithms described above as being implemented by the image-based tracking module 107, can be utilized to visually track each NIR marker in the image. The 3D estimate of a marker position can be found by triangulation of the NIR marker image as seen in both left 701 and right 703 NIR stereo image pairs. The 3D estimate of the NIR marker can then be re-projected as an overlay in the RGB image 702. The tool position can also be found from the stereo image pair. The stereo NIR system can be replaced by a 3D sensing camera capable of NIR observation.

The embodiments described herein can also be applied in non-stereo applications. For example, the system can be implemented for mono camera applications. For manual and master-slave modes (FIG. 2), mono camera images are sufficient. In semi-autonomous mode, however, depth of the target points is important for the robot to perform positioning tasks, making stereo imaging critical to provide depth information. There are other depth sensors, however, that do not require a second camera, such methods employing time of flight, conoscope, laser, and other depth cameras. This invention would also work with single cameras for manual and master-slave mode. For semi-autonomous mode, the present embodiments would also work with single camera and an additional depth sensor.

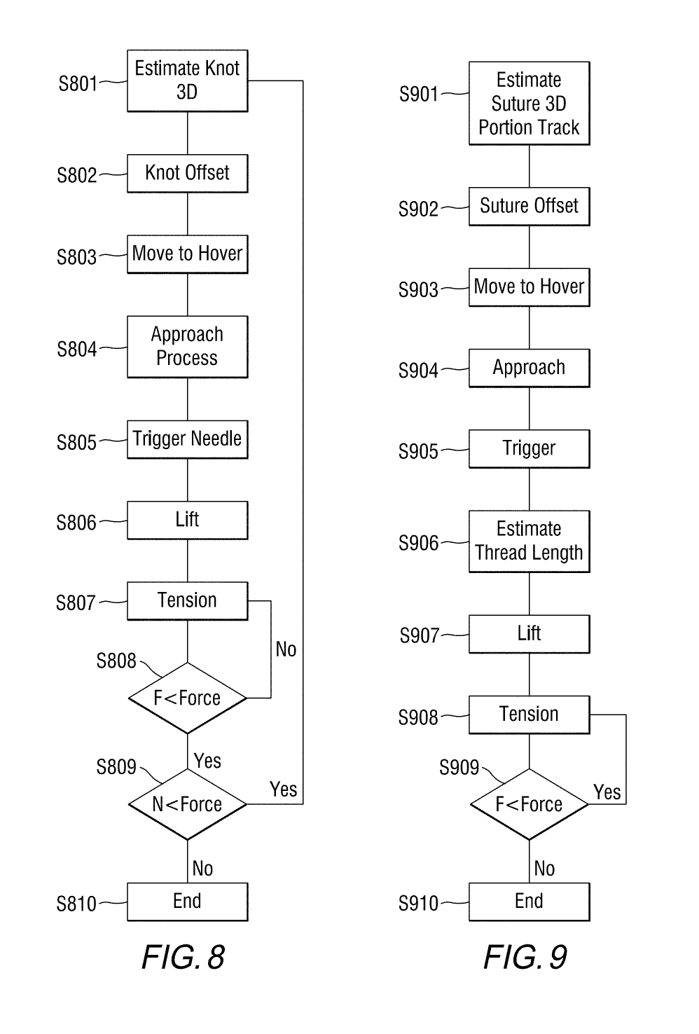

FIG. 8 and FIG. 9 illustrate two flow charts of exemplary robotic operation algorithms implemented by the system. For instance, FIG. 8 illustrates an algorithm for robotic knot tying and FIG. 9 illustrates an algorithm for robotic suturing. The marker positions are used to estimate knot 3D position (FIG. 8) and suture 3D position (FIG. 9). The flow charts describe the robotic motions that follow position estimation.

As is shown in FIG. 8, the robotic operation algorithm begins in step S801 with the execution of an estimation of the knot. In step SS02, the knot offset is determined and communicated to the robot. In step S803, the robot moves to hover above the suture placement. In step S804, the approach process is performed. In the approach process, the robot takes into account the position information obtained based on the detected markers. Thus, the robot uses visual servoing to guide the needle toward the NIR marker. In step, S805 the needle is triggered. This trigger could be met when the robot has come within a predetermined distance of the knot. In step S806, the robot lifts the tool to pull enough thread. In step S807, the robot lifts the tool furthermore until a sufficient tension F is measured in the thread. This process is repeated for the number of desired loops in the knot.

FIG. 9 is an example of a robotic suturing process. In step S901, the suture 3D position track is estimated. In step S902, the suture offset is determined. In step S903, the robot moves to hover above the suture placement. In step S904, the robot uses visual servoing to drive the needle toward the placement indicated by the NIR marker. In step S905, the suture is triggered. In step S906, an estimation of the length, of thread is calculated. Using this estimation, in step S907, the robot lifts the needle to complete the suture. In steps S908, S909, the robot lifts the needle until a tension of F is measured in the thread. The system exits if the tension is greater than F.

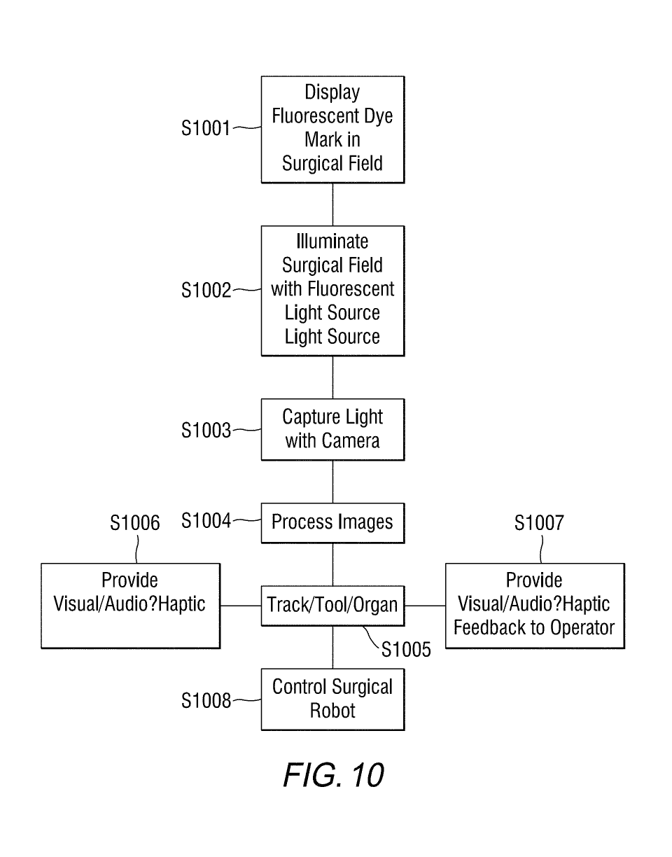

FIG. 10 illustrates an overall process according to one embodiment. In step S1001, fluorescent dye markers are deployed to a surgical field. The dye markers can be deployed, for example, by spraying, painting, attachment, tissue injection, intravenous injection, and the like. In step S1002, the surgical field can be illuminated with fluorescent and visible light sources. In step S1003, light can be captured with a camera. The light captured by the camera may include light emitted within both the visible range and IR range. In step S1004, the resulting images can be processed by the image processing algorithms described previously in order to identify markers in the image. In step S1005, based on the detected markers, the tool or organ, which is marked by the markers, can be tracked. This tracking is described in detail previously and includes determining the location of tools, organs, or other marked portions of the subject within the surgical field based on markers which are associated with respective elements. In step S1006, a stereo display can be provided based on the tracking. In step S1008, visual, audio and haptic feedback can be provided to the surgeon. In step S1009, a robot can be controlled based on the tracking.

Certain portions or all of the disclosed processing, such as the image processing and visual tracking algorithms, for example, can be implemented using some form of computer microprocessor. As one of ordinary skill in the art would recognize, the computer processor can be implemented as discrete logic gates, as an Application Specific Integrated Circuit (ASIC), a Field Programmable Gate Array (FPGA) or other Complex Programmable Logic Device (CPLD). An FPGA or CPLD implementation may be coded in VHDL, Verilog or any other hardware description language and the code may be stored in an electronic memory directly within the FPGA or CPLD, or as a separate electronic memory. Further, the electronic memory may be non-volatile, such as ROM, EPROM, EEPROM or FLASH memory. The electronic memory may also be volatile, such as static or dynamic RAM, and a processor, such as a microcontroller or microprocessor, may be provided to manage the electronic memory as well as the interaction between the FPGA or CPLD and the electronic memory.

Alternatively, the computer processor may execute a computer program including a set of computer-readable instructions that perform the functions described herein, the program being stored in any of the above-described non-transitory electronic memories and/or a hard disk drive, CD, DVD, FLASH drive or any other known storage media. Further, the computer-readable instructions may be provided as a utility application, background daemon, or component of an operating system, or combination thereof, executing in conjunction with a processor, such as a Xenon processor from Intel of America or an Opteron processor from AMD of America and an operating system, such as Microsoft VISTA, UNIX, Solaris, LINUX, Apple, MAC-OSX and other operating systems known to those skilled in the art.

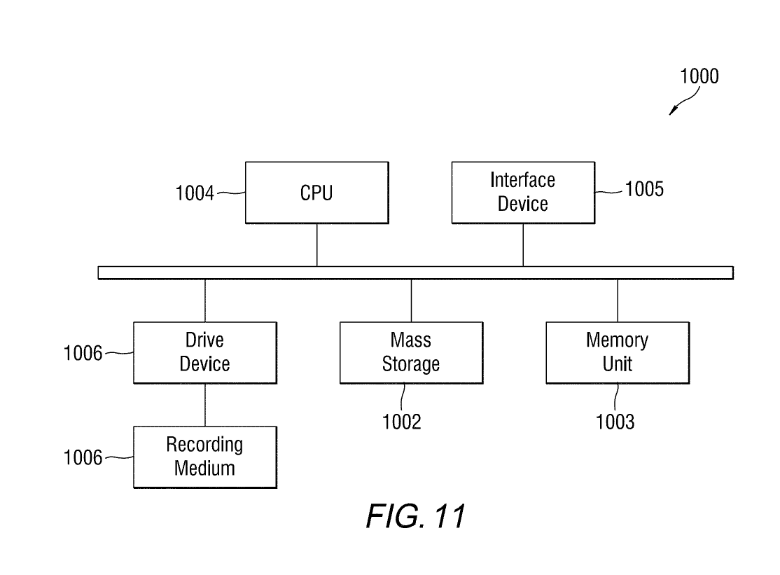

In addition, certain features of the embodiments can be implemented using a computer based system (FIG. 11). The computer 1000 includes a bus B or other communication mechanism for communicating information, and a processor/CPU 1004 coupled with the bus B for processing the information. The computer 1000 also includes a main memory/memory unit 1003, such as a random access memory (RAM) or other dynamic storage device (e.g., dynamic RAM (DRAM), static RAM (SRAM), and synchronous DRAM (SDRAM)), coupled to the bus B for storing information and instructions to be executed by processor/CPU 1004. In addition, the memory unit 1003 may be used for storing temporary variables or other intermediate information during the execution of instructions by the CPU 1004. The computer 1000 may also further include a read only memory (ROM) or other static storage device (e.g., programmable ROM (PROM), erasable PROM (EPROM), and electrically erasable PROM (EEPROM)) coupled to the bus B for storing static information and instructions for the CPU 1004.

The computer 1000 may also include a disk controller coupled to the bus B to control one or more storage devices for storing information and instructions, such as mass storage 1002, and drive device 1006 (e.g., floppy disk drive, read-only compact disc drive, read/write compact disc drive, compact disc jukebox, tape drive, and removable magneto-optical drive). The storage devices may be added to the computer 1000 using an appropriate device interface (e.g., small computer system interface (SCSI), integrated device electronics (IDE), enhanced-IDE (E-IDE), direct memory access (DMA), or ultra-DMA).

The computer 1000 may also include special purpose logic devices (e.g., application specific integrated circuits (ASICs)) or configurable logic devices (e.g., simple programmable logic devices (SPLDs), complex programmable logic devices (CPLDs), and, field programmable gate arrays (FPGAs)).

The computer 1000 may also include a display controller coupled to the bus B to control a display, such as a cathode ray tube (CRT), for displaying information to a computer user. The computer system includes input devices, such as a keyboard and a pointing device, for interacting with a computer user and providing information to the processor. The pointing device, for example, may be a mouse, a trackball, or a pointing stick for communicating direction information and command selections to the processor and for controlling cursor movement on the display. In addition, a printer may provide printed listings of data stored and/or generated by the computer system.

The computer 1000 performs at least a portion of the processing steps of the invention in response to the CPU 1004 executing one or more sequences of one or more instructions contained in a memory. such as the memory unit 1003. Such instructions may be read into the memory unit from another computer readable medium, such as the mass storage 1002 or a removable media 1001. One or more processors in a multi-processing arrangement may also be employed to execute the sequences of instructions contained in memory unit 1003. In alternative embodiments, hard-wired circuitry may be used in place of or in combination with software instructions. Thus, embodiments are not limited to any specific combination of hardware circuitry and software.

As stated above, the computer 1000 includes at least one computer readable medium 1001 or memory for holding instructions programmed according to the teachings of the invention and for containing data structures, tables, records, or other data described herein. Examples of computer readable media are compact discs, hard disks. floppy disks, tape, magneto-optical disks, PROMs (EPROM, EEPROM, flash EPROM), DRAM, SRAM, SDRAM, or any other magnetic medium, compact discs (e.g., CD-ROM), or any other medium from which a computer can read.

Stored on any one or on a combination of computer readable media, the present invention includes software for controlling the main processing unit 1004, for driving a device or devices for implementing the invention, and for enabling the main processing unit 1004 to interact with a human user. Such software may include, but is not limited to, device drivers, operating systems, development tools, and applications software. Such computer readable media further includes the computer program product of the present invention for performing all or a portion (if processing is distributed) of the processing performed in implementing the invention.

The computer code elements on the medium of the present invention may be any interpretable or executable code mechanism, including but not limited to scripts, interpretable programs, dynamic link libraries (DLLs). Java classes, and complete executable programs. Moreover, pans of the processing of the present invention may be distributed for better performance, reliability, and/or cost.

The term “computer readable medium” as used herein refers to any medium that participates in providing instructions to the CPU 1004 for execution. A computer readable medium may take many forms, including but not limited to, non-volatile media, and volatile media. Non-volatile media includes, for example, optical, magnetic disks, and magneto-optical disks, such as the mass storage 1002 or the removable media 1001. Volatile media includes dynamic memory, such as the memory unit 1003.

Various forms of computer readable media may be involved in carrying out one or more sequences of one or more instructions to the CPU 1004 for execution. For example, the instructions may initially be carried on a magnetic disk of a remote computer. An input coupled to the bus B can receive the data and place the data on the bus B. The bus B carries the data to the memory unit 1003, from which the CPU 1004 retrieves and executes the instructions. The instructions received by the memory unit 1003 may optionally be stored on mass storage 1002 either before or after execution by the CPU 1004.

The computer 1000 also includes a communication interface 1005 coupled to the bus B. The communication interface 1004 provides a two-way data communication coupling to a network that is connected to, for example, a local area network (LAN), or to another communications network such as the Internet. For example, the communication interface 1005 may be a network interface card to attach to any packet switched LAN. As another example, the communication interface 1005 may be an asymmetrical digital subscriber line (ADSL) card, an integrated services digital network (ISDN) card or a modern to provide a data communication connection to a corresponding type of communications line. Wireless links may also be implemented. In any such implementation, the communication interface 1005 sends and receives electrical, electromagnetic or optical signals that carry digital data streams representing various types of information.

The network typically provides data communication through one or more networks. to other data devices. For example, the network may provide a connection to another computer through a local network (e.g., a LAN) or through equipment operated by a service provider, which provides communication services through a communications network. The local network and the communications network use, for example, electrical, electromagnetic, or optical signals that carry digital data streams, and the associated physical layer (e.g., CAT 5 cable, coaxial cable, optical fiber, etc). Moreover, the network may provide a connection to a mobile device such as a personal digital assistant (PDA) laptop computer, or cellular telephone.

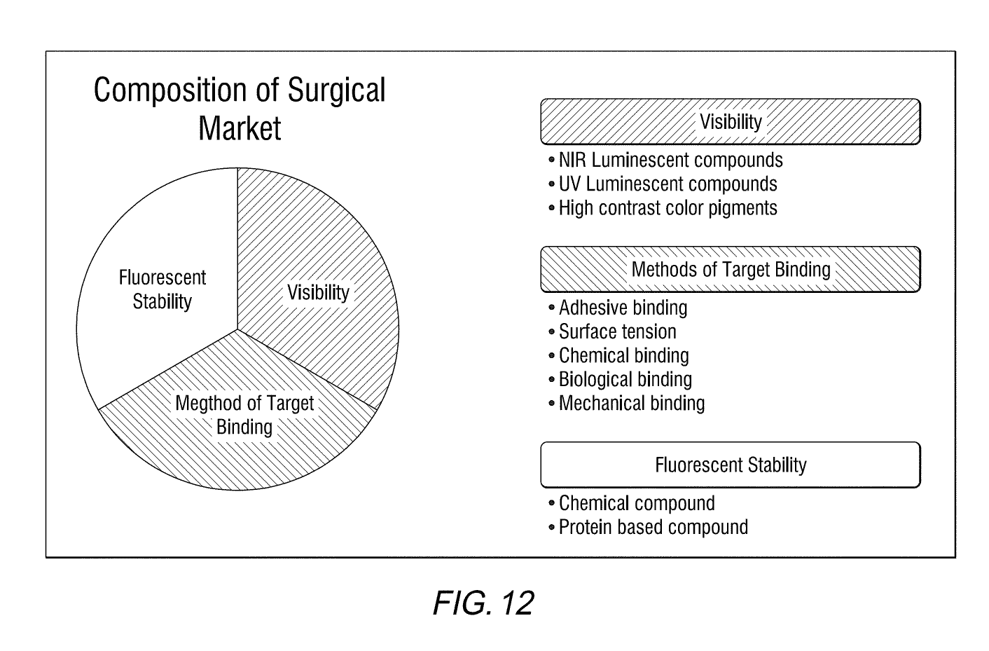

Returning to a fundamental description of the present disclosure, FIG. 12 is a schematic of factors that should be considered when development a fluorescent marker, or marker. For instance, a marker may be comprised of two or more components that make it suitable for use in a surgical field. Critically, the components should be considered according to their specific qualities as it relates to visibility, target binding, and fluorescent stability. The marker may contain any ratio of any combination of the components described in FIG. 12. Subsequent discussion refers to exemplary embodiments of the marker composition, but it should be appreciated that any composition of marker may be considered in order to provide the visualization described herein. In an exemplary embodiment, a marker may include, at least, 0.1 mg ICG, 0.5 mL acetone, and 0.25 mL cyanoacrylate.

In order to be used in the surgical field, a marker should be robust to occlusion from collateral tissue, blood, and smoke, among other surgical conditions, so as to be visible. The marker should also be easily segmented from surrounding tissue, such that it is distinguishable from natural features in the surgical field and can be tracked. Additionally, the marker should be stable such that it can be visualized for an entirety of a procedure. In the exemplary embodiment described above, a marker can be made from, at least, near infrared (NIR) fluorophore such as indocyanine green (ICG). The use of ICG is favorable for three reasons. First, ICG is an FDA approved drug that is routinely administered in a number of surgeries, including cardiac, procedures as a contrast agent. Second, the fluorescence of ICG is in the NIR spectrum allowing it to be easily segmented from sounding features in a NIR image. Third, once activated, the fluorescence of ICG will remain stable for multiple hours allowing it to be used in most surgical procedures. It can be appreciated that the use of ICG is merely exemplary of a variety of NIR fluorescent compounds that could be used in creating a marker. For instance, the NIR fluorescent compound may be Prussian blue. In addition to the near infrared spectrum, the marker may contain compounds that allow fluorescence in other spectra, as well. For instance, an ultraviolet (UV) fluorescent compound may have similar benefits in the surgical field as the NIR compounds discussed above. Fluorescence in the UV range would allow the markers to be segmented and tracked for the entirety of the surgical procedure, including instances when the marker may be occluded by blood or surrounding tissue. Additionally, the marker may contain a compound that improves visibility of the marker in the visible range. One example of this technique would be to add a bluish/cyan pigment, such as methyl blue, to the marker composition. While visualization is not as robust when occluded by blood or tissue in the surgical field, the pigment would create high contrast between the marker and tissue which would allow it to be segmented from the background.

In addition to visibility, as shown in FIG. 12, it is important for a surgical marker to contain a compound to bind to target tissue. When placed in the surgical field, it may be desirable for a marker to remain at its initial placement. Fixating the marker to tissue can be a difficult task as the surgical field maybe highly deformable, obstructed, irrigated, or a combination thereof. In the exemplary embodiment described with respect to FIG. 12, the marker includes a cyanoacrylate to achieve sufficient adhesion for the marker to remain fixed to tissue for the entirety of a surgical procedure. Cyanoacrylate has the advantage of being an FDA approved compound for some medical procedures, such as wound closure on superficial tissue. Additionally, cyanoacrylate will polymerize when in contact with water, allowing the marker to harden in the applied shape as it fuses to tissue. Finally, cyanoacrylate is a highly viscous chemical, which allows the user fine control over the amount of solution applied for each marker. In another embodiment, the mechanism to control binding of the marker may consist of at least one of the following: surface tension, adhesion, chemical/biological, and mechanical binding, as binding of the marker to target tissue can be accomplished through compounds that increase surface tension. In an embodiment, an additional compound can be polyethylene glycol (PEG). The addition of PEG into the marker composition makes the marker more viscous, which allows it to be placed more consistently in an irrigated surgical field. In an embodiment, cyanoacrylate could be replaced in the marker with the use of a biocompatible epoxy. The epoxy may be selectively curable with the addition of UV or NIR light. The marker composed of biocompatible epoxy would have the advantage of remaining fluid when mixed and applied to target tissue. and binding to target tissue on command.

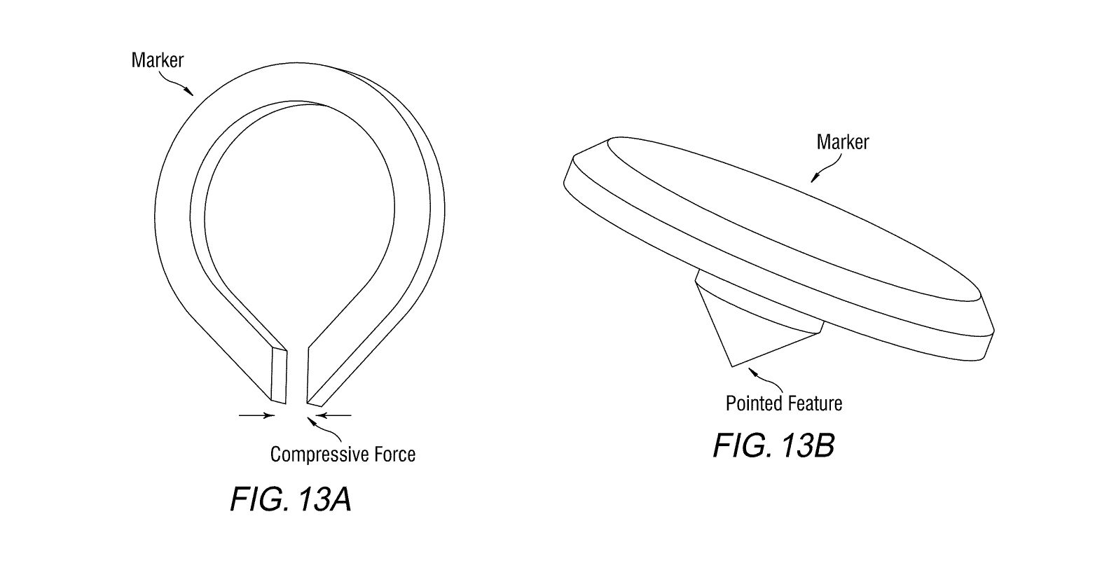

In an embodiment, the marker may contain a compound that chemically or biologically binds to the target tissue. In one example, the marker may contain an algae compound that binds and creates a fibrous network when applied to a cut edge of tissue. In another embodiment, the marker may contain a coagulant compound such as aluminum sulfate that chemically binds to a bleeding edge of tissue. In another embodiment, the marker may contain a compound that can selectively bind to any number of proteins that are exposed along a cut edge of tissue. Finally, the marker may be designed such that it mechanically binds to target tissue. In these embodiments, it is assumed that the marker is applied to the target tissue in a solid state. In an embodiment, and as shown in FIG. 13A, the marker may be formed such that it is biased to apply compressive force to a tissue edge. The compressive force pinches target tissue and allows the marker to remain in place. In another embodiment, as shown in FIG. 13B. the marker may contain a sharp, or pointed, feature capable of piercing target tissue for fixation. In another embodiment, the marker may contain a compound such as silicone that allows the marker to be molded in any arbitrary shape. The addition of silicone would allow the stiffness of the marker to be varied from soft to hard, make the marker biologically inert, and allow the marker to stretched and shaped for any application.

According to an embodiment, and depending on the target location, it may be desirable to enhance the visualization of the surgical markers. In the exemplary embodiment described with respect to FIG. 12, the marker may include acetone, which can react with ICG to increase NIR fluorescence and can react with cyanoacrylate to polymerize the marker in the fluoresced state. In an additional embodiment of the marker, the composition may include water which will behave similarly in enhancing the fluorescence but has the additional property of acting as a catalyst in polymerization of the marker solution. In another embodiment, the marker may include ethanol which behaves similarly to water but delays the polymerization time such that the time a user has to apply the marker in the surgical field is increased. In still another embodiment, the marker solution may contain a protein such as bovine serum albumin (BSA). As with other compounds, when BSA binds with ICG, it acts to enhance fluorescence. One advantage of BSA is that it will not affect the polymerization time of the marker.

The combination of the fluorescent, binding, and stability compounds described with respect to FIG. 12 and discussed above with respect to the exemplary embodiment form the fluorescent markers described in the present disclosure. Listed below are preferred combinations of these compounds to produce stable, fluorescent markers. It can be appreciated that this list is not exhaustive but intended to illustrate examples of the preferred disclosure:
ICG+water+cyanoacrylate
ICG+ethanol+cyanoacrylate
ICG+acetone+cyanoacrylate
ICG+PEG+water
ICG+PEG+acetone
ICG+PEG+ethanol
ICG+alge(Vetigel)
ICG+coagulant(styptic pen/aluminum oxide)
ICG+biocompatible epoxy+acetone

According to an embodiment, the shape of a marker can affect functionality in its application. For instance, a small circular marker may be ideal for tracking the motion of a point of interest while a linear marker may be ideal for tracking the boundary of diseased tissue. Further still. an irregular marker may be ideal for separating background from fore ground tissue. A surgical marker according to the present disclosure can be applied to target tissue in one of two states, either as a liquid or as a solid. In the event that a liquid marker is applied to the surgical site, it may remain a liquid for the duration of the procedure, functionally bind to the surgical site, or undergo a chemical reaction to solidify on the target tissue.

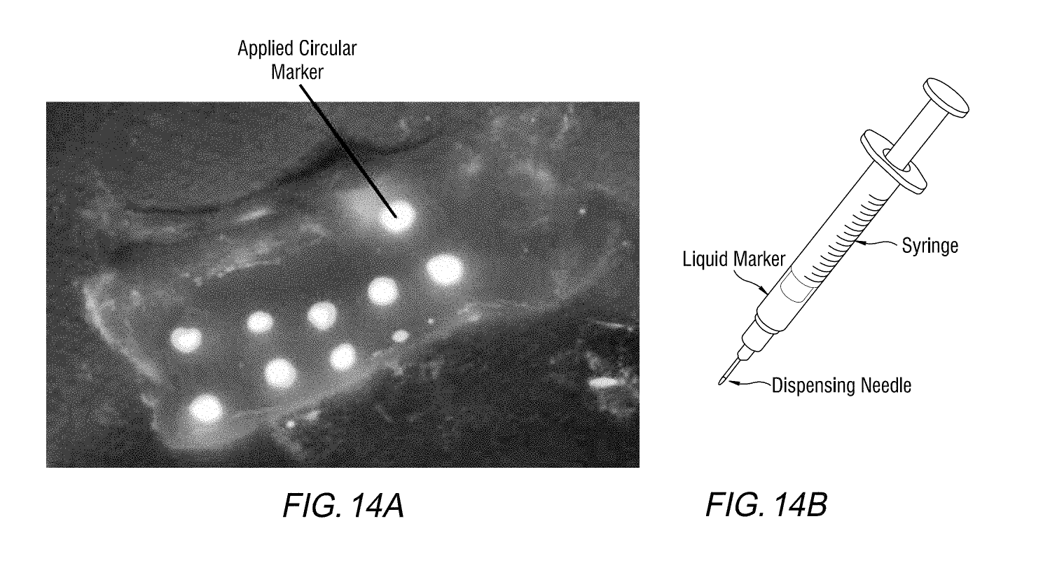

In an embodiment, and as shown in FIG. 14A and FIG. 14B, the marker can be applied to target tissue in a liquid state through a syringe needle. Applying a liquid marker to target tissue has a number of advantages. First, the marker can be mixed onsite which improves the stability of the marker. Second, a liquid marker allows the precise control over location and application to target tissue. Third, the marker can be applied as any irregular shape. By applying a liquid marker with syringe, the irrigated surgical field initiates an exothermic reaction to solidify the marker in a circular shape to target tissue. A circular marker may be beneficial for tracking single points of interest on target tissue during a surgical procedure.

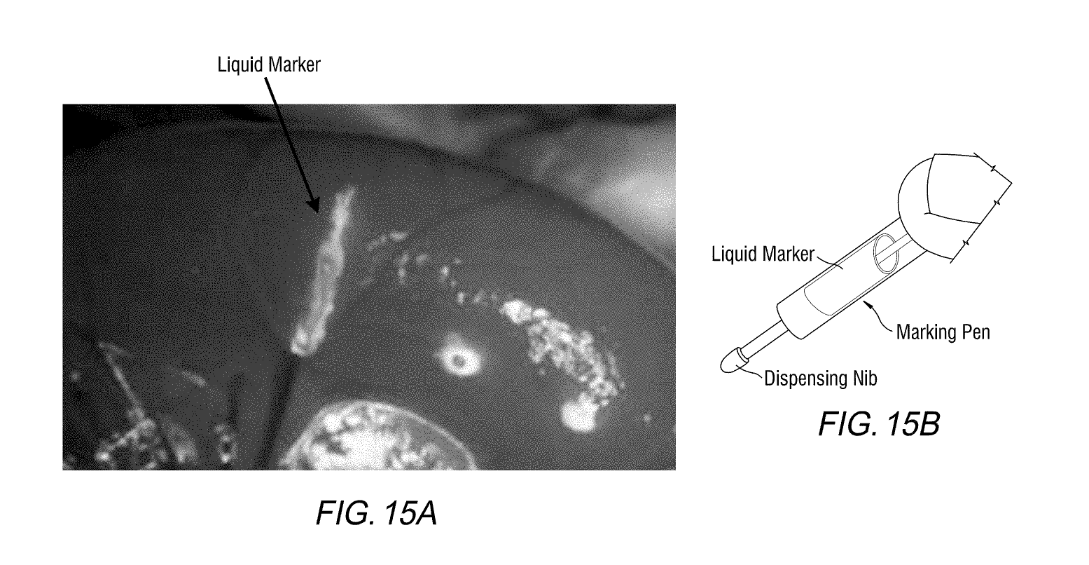

In an embodiment, and as shown in FIG. 15A and FIG. 15B, a marking tip such as a syringe needle or felt nib may be used to dispense the marker in a linear pattern. By applying the marker as a continuous line, one can use the marker to define boundaries on target tissue. Defining boundaries may be useful to identify regions of diseased tissue or regions where a surgical procedure should not be performed.

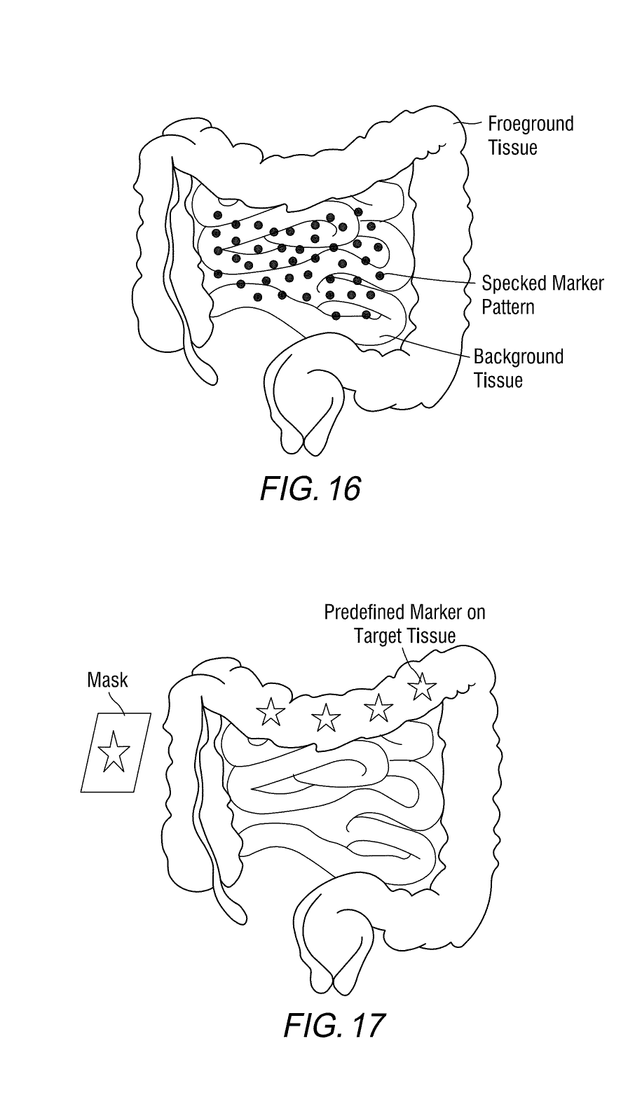

In an embodiment, and as shown in FIG. 16, the liquid marker may be sprayed onto the target tissue to create a speckled pattern when polymerized. A speckled pattern may be of interest to define lark regions of tissue from each other. In the example illustrated in FIG. 16, background tissue may be speckled to distinguish it from foreground tissue. Other components in robotic or semi-autonomous workflow may use background and foreground information to plan or control their motions or suggestions.

Similarly, in an embodiment and as shown in FIG. 17, the liquid marker may by applied though a predefined mask to apply the marker in any arbitrary and predefined shape on target tissue. As illustrated in FIG. 17, a liquid marker delivered to the target tissue as an aerosol directed at a mask can create mask-shaped markers on the tissue of interest.

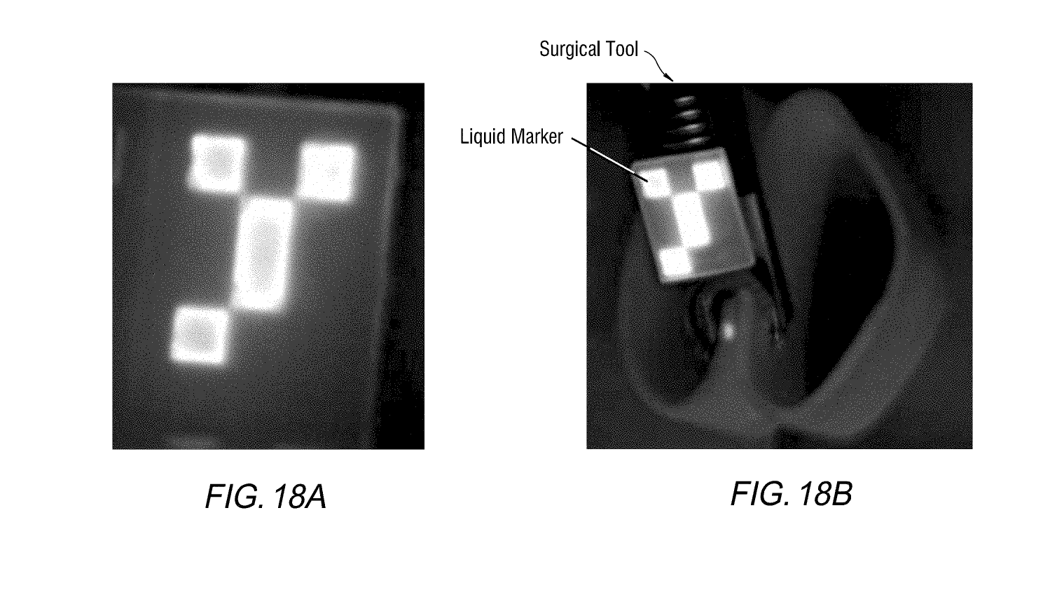

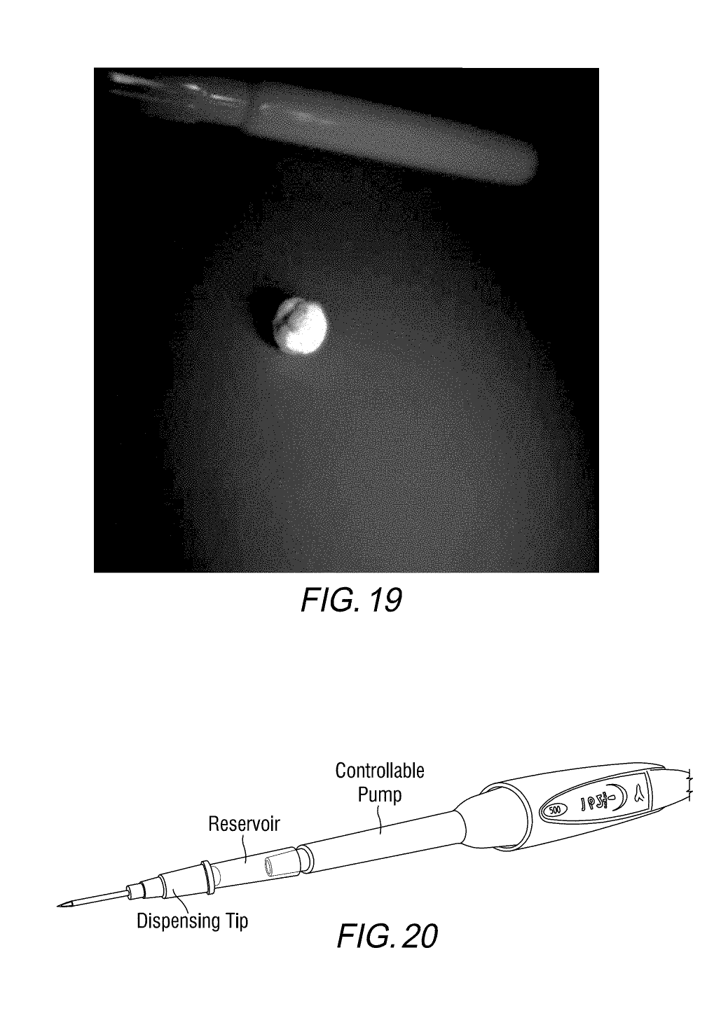

According to an embodiment, the surgical markers may be applied to the target tissue in the solid state. Applying a solid marker to the surgical field has the advantage of predefining and creating a marker shape with tight and repeatable tolerances. Additionally, a solid marker may be more robust in maintaining a predefined shape for the duration of a surgical procedure. FIG. 18A, and FIG. 18B, for instance, illustrate an exemplary embodiment of a predefined solid marker shaped into a checkered pattern. A robust checkered pattern. may be useful for tracking the location of tool in the surgical field of view. The pattern may be assembled as a series of wells that are filled with polymerized liquid markers, machined from a polymerized liquid marker, or molded from a polymer such as silicone. A fluorescent silicone marker would have the advantage of variable stiffness throughout the dimensions of the component and may be molded into any arbitrary shape, including a cylindrical sleeve to be attached to surgical any tool. FIG. 19, for instance, illustrates a NIR fluorescent silicone component (i.e., white sphere in image) that was molded into a ball.

According to an embodiment, the solid markers can be manufactured into any arbitrary and predefined shape through standard injection molding with plastic and silicone polymers, for instance. As described in FIG. 13A and FIG. 13B, the solid marker may be in the form of a clip or have a sharp edge capable of mechanically fixating the solid marker to target tissue. The solid marker may be dissolvable. such that it mechanically fixates to tissue permanently and is absorbed by the body over time. In another embodiment, the solid marker may be removable such that the mechanical fixation is temporary and can be removed at the completion of the procedure. A solid surgical marker may be disposable at the end of a surgical procedure or sterilizable for use in other procedures.

Of equal import to the composition and shape of the surgical marker, an apparatus used to apply the surgical marker will now be described. The marking apparatus can be of a variety of forms including, for example, a tool used for marking target tissue in open surgical procedures and a tool designed to mark tissue for laparoscopic surgical tasks. In addition, features of the marking tool that allows components of a surgical marker to be mixed prior to application are disclosed.

FIG. 20 shows an exemplary embodiment of a marking apparatus for applying markers in the open surgical setting. The marking tool consists of controllable pump, solution reservoir, and dispensing needle. The reservoir and needle can be permanently fused. To use the tool, the reservoir can be selectively removed from the pump and filled with the liquid marker. The reservoir can be reattached to the pump and positioned over the target tissue. To dispense the liquid marker, the handle of the pump can be twisted, thereby increasing the pressure inside of the reservoir. The pressure can be increased until the tool dispenses the desired volume of liquid marker on the target tissue. The dispensing tip can be sized such that the liquid marker is dispensed at the correct speed and with the correct diameter. Also, the dispensing tip may be swapped for a nib, tube, or other device that would allow dispensing of the marker in an alternate pattern such as a line, speckle, or other arbitrary shape. The system of FIG. 20 may also be used for laparoscopic marking as the tool is small enough to fit though a surgical port. In an alternative embodiment, the pump can be mechanized to dispense the liquid markers in a programmed volume. The dispensing tip can be lengthened further to reach deep tissue laparoscopically, such as the colon stump, pelvic wall, and vaginal tissues. In another embodiment, the dispensing tool can be mounted onto a robotic arm that can be programmed to dispense the markers within the surgical field through manual, semi-autonomous, or autonomous control of the robot.

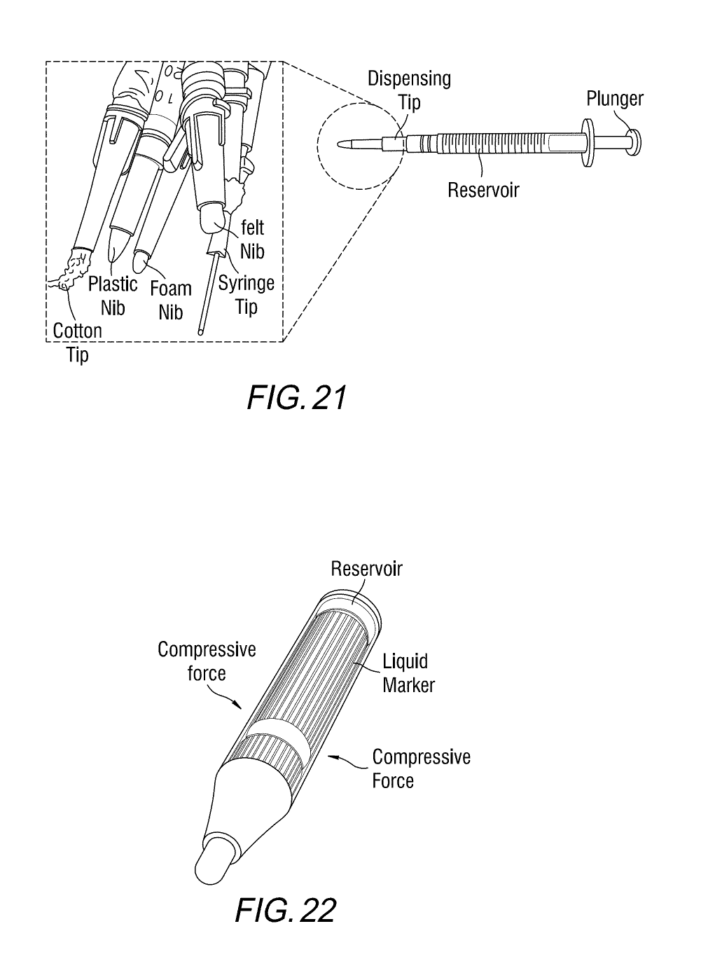

FIG. 21 illustrates an alternative embodiment of an open surgical marking tool. The marking tool is similar to FIG. 20 with two exceptions. First, the mechanism to dispense the liquid marker is activated by compression of a plunger at the rear of the device. While this mechanism makes dispensing a known volume more difficult, the entire device can be disposed after application of the marker. Second, because the design is disposable, the reservoir can be prefilled will the liquid marker solution. This allows the device to be manufactured and packaged in a single assembly guaranteeing sterility for surgical use. To use, a user need only to remove the tool from sterile packaging and dispense to apply the liquid marker onto the target tissue. Similar to FIG. 20, a number of dispensing tips can be packaged with the device to dispense various mark types and sizes such as, among others, cotton, felt, plastic, tubular, and metal. The marking tool according to FIG. 21 can be used for laparoscopic procedures as it is long enough to pass through a laparoscopic port to the site of interest.

In an embodiment, the marking apparatus may dispense liquid marker by an ink pen mechanism with felt tip, cotton nib, foam nib, or other suitable tip. An advantage of this method is that the shape and size of the marking may be controlled by hand. It may be particularly useful for segmentation tasks and automation, such as drawing “no-fly zones” or drawing lines along which to cut.

In another embodiment, a liquid marker may be dispensed to the target tissue by application of a compressive force to the outside of the tool. As shown in FIG. 22, a marking tool may comprise a compliant reservoir, a liquid marker, and a dispensing tip. The liquid marker may be contained within an ampule such that it does not leak from the dispensing tip until the user is ready to apply the liquid marker. By squeezing the outside of the reservoir, the user is able to rupture an internal ampule releasing the liquid marker into the reservoir. Additional pressure on the reservoir forces the liquid marker through the attached dispensing nib and onto the target tissue. As with other methods, the shape of applied marker may be varied by changing the style of the dispensing tip.

In an embodiment, the marker components may be separated into distinct ampules prior to their use, then shaken together to mix before application, thereby ensuring components that are volatile retain activity before application.

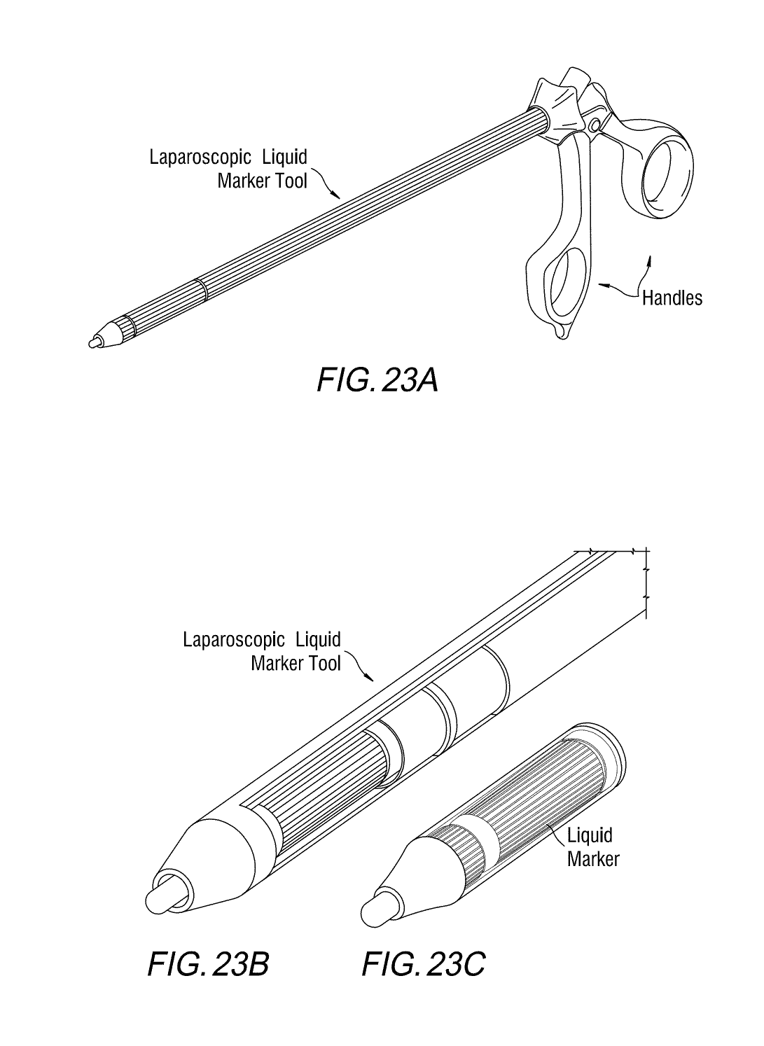

Additionally, the dispensing tool may be compatible with a laparoscopic hand tool for use in a laparoscopic surgery. FIG. 23A and FIG. 23B illustrates an approach in which an apparatus for open surgical marking can be combined with a laparoscopic tool for internal surgery. In an embodiment, a marking tool, as shown in FIG. 23B, can be inserted into the laparoscope of FIG. 23A as a cartridge. The open surgical tool may then be loaded as a single assembly. When the handles of the laparoscopic hand tool are squeezed together, pressure is transmitted down the laparoscopic tool and applied to the conformable reservoir. By increasing the pressure on the outside of the reservoir, the liquid marker will be forced out of the dispensing tip and onto the target tissue, as similarly described previously.

In another embodiment, the dispensing tool may be combined with traditional surgical tools in order to facilitate relevant marking during a procedure. For example, a pair of surgical scissors may be modified so that they automatically dispense ICG along the cut edge, perhaps for tracking and later sewing together the new edges after a resection procedure. In another embodiment, a stapler may be configured such that it automatically. dispenses ICG along the stapled edge. applicable for a myriad of purposes including tracking and tissue analysis.

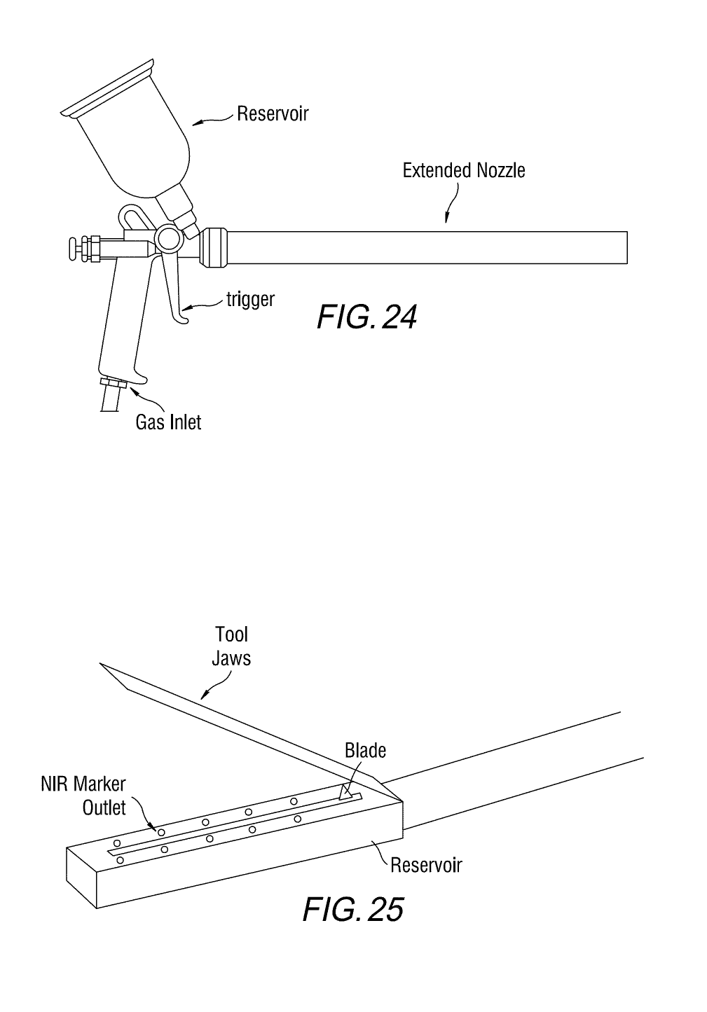

In another embodiment, the dispensing tool may be a device capable of spraying the liquid marker on the target tissue in a fine mist. This device could be a spray bottle configured for dispensing a precise amount of liquid marker, or a pressurized gun applicator allowing control of the rate of liquid application. By spraying target tissue. one may be able to mark large areas of tissue with a speckled pattern. FIG. 24, for instance, illustrates one approach to this surgical tool for an open procedure. The liquid marker may be stored within the reservoir of the tool. An inlet controlling the flow of gas may be coupled to a trigger, lever, or other mechanism that can be activated by the user. When the user is ready to apply the liquid marker to target tissue, the inlet can be opened and the tool can be pressurized. As the tool builds pressure, the gas mixes with the liquid marker and is forced out of the dispensing nozzle onto the target tissue. The dispensing nozzle may be lengthened such that the apparatus can be fit through a surgical port for laparoscopic surgery. Additionally, the shape of the dispensing tip can be altered to change the pattern of the applied marker. Alternatively, the dispensing tip may contain a mask to spray in an arbitrary or predefined pattern, as similarly described with reference to FIG. 17.

FIG. 25 illustrates the delivery tip of another embodiment of a surgical marking apparatus for both liquid and solid markers. In this concept, liquid surgical markers are loaded into the reservoir on the surgical tool. The reservoir can hold liquid marker to be applied through the dispensing nozzle. In an embodiment, the reservoir can be replaced by a clip containing solid markers.

With reference to FIG. 25, the tool may have three states depending on the pressure applied to the tool handle. When the handle is first squeezed, the jaws of the tool close around a target tissue, pinching the target tissue between jaws of the tool. If additional pressure is applied to the tool handle, the liquid markers are forced from the reserve, through the tool, and out of the dispensing tips in the jaw of the tool. The pattern the liquid markers are dispensed in may be varied depending on the shape of the dispensing tips. At this point, the user can release pressure from the tool handle, leaving the marked tissue in the surgical site. Alternatively, a user could engage a third state of the tool by applying additional pressure to the handle. The third state pushes a blade lengthwise across the jaws of the tool. The blade is intended to separate the target tissue into two regions, both regions being equally marked. This strategy may be useful for surgical procedures that require entire tissue edges to be marked, such as with bowel anastomosis. In an alternative embodiment, the marking tool may contain a cartridge filled with solid markers. When pressure is applied to the handle of the tool, the solid markers are dispensed to the target tissue.

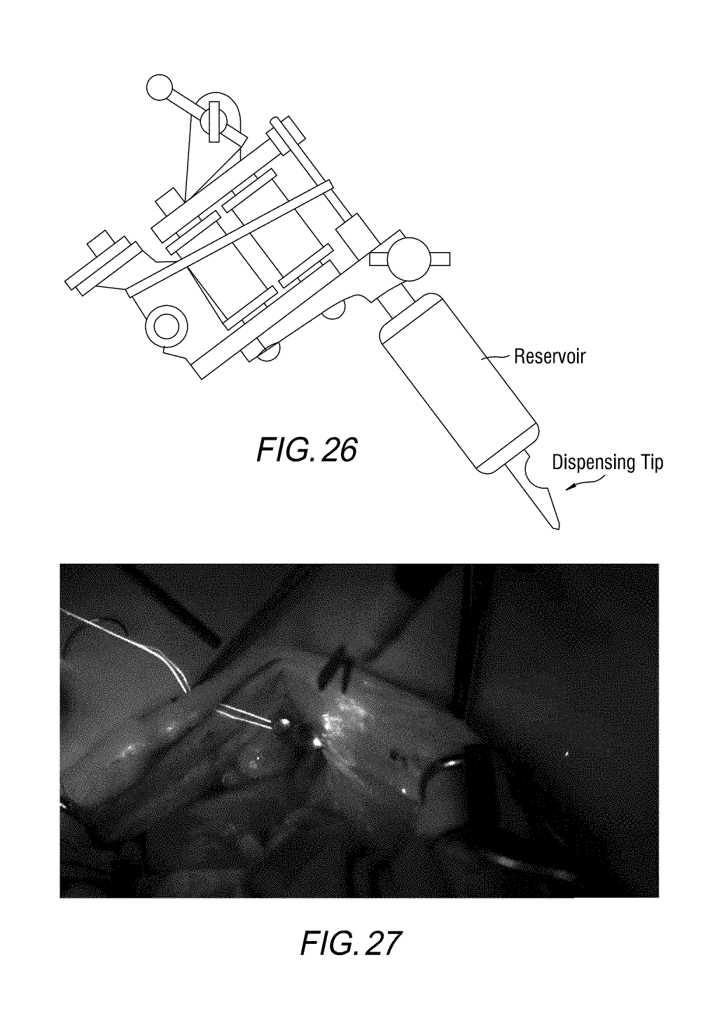

In another embodiment, the marking apparatus may be used to embed liquid or solid markers in or beneath a superficial layer of target tissue. FIG. 26, for instance, illustrates how a tattoo gun may be modified to apply the surgical markers to soft tissue. In this embodiment, liquid markers are placed within a reservoir near the dispensing tip of the tool. The marking tool is then placed over target tissue and powered on. When the marking tool is activated, the dispensing tip oscillates, piercing the target tissue multiple times. With each piercing, the dispensing nib releases a liquid marker into the target tissue. In another embodiment, a syringe may be loaded with solid or liquid markers. The tip of the syringe may be outfitted with a hypodermic needle. The user can pierce just below the superficial layer of target tissue and inject the surgical marker into the tissue.

In addition to using an application tool, one could develop a system to deliver the marker to target tissue using biological process in the body. In one embodiment, the marker could be encapsulated in a nanoparticle and injected into the periphery of the patient. The nanoparticle may contain a structure capable of binding to receptor sites at the target tissue. The markers would travel through the body until they reach the target binding, sites. Alternatively, instead of injecting the marker into the patient, the markers could be encapsulated and ingested by the patient as a liquid or pill. After the marker is ingested, it can be absorbed by the blood stream and distributed throughout the body. In an alternative embodiment, the markers may be encapsulated in a structure capable of binding to proteins present at the surgical site. The proteins may be inherent to target tissue or present because of a surgical task. Such proteins can include proteins for coagulation after a cut or proteins embedded in tissue layers that are exposed after a cut. The markers could then be applied to the entire surgical scene and selectively bind to target tissue sites.

In another embodiment, a mask or stencil could be used to allow marking in a predefined region. This could allow differentiation between marker types, 2D/3D registration and tracking of specific markers and analysis of the deformation of underlying tissue. For example, the deformation of the heart could be imaged using video cameras and, after a single dispersion of marker through a calibrated stencil, the motions of the heart could be quantified and the underlying 3D geometry known, in a technique similar to structured light approaches.

In a similar embodiment to the stencil approach mentioned previously, precise marker geometry could be molded and used in a similar fashion for tracking and deformation estimation. It could be used for tool or tissue tracking applications, as reference markings for a manual or robotic surgical procedure, or as a way of precisely defining different, easily differentiable markers to allow segmentation of features based on marker type. For example, tool tracking applications may prefer a premolded geometry which allows 3D tracking and registration.

Another embodiment of the present disclosure includes a NIRF clip hooked into tissue providing tracking while possibly simultaneously holding tissue in place. NIRF clips could be used similarly to optical trackers but with the advantages described above. including resistance to occlusion. Clips could be disposable and/or dissolvable or removed after the procedure is finished.

In another embodiment, and as illustrated in FIG. 27, the markers can be applied to a section of surgical suture. or along the entire length of the suture. NIR-coated suture could be used as part of a surgical procedure and would remain fluorescent for the procedure. This would be useful as distinguishing suture from the background can be difficult due to its thin size and lack of contrast. The suture can be made from any biocompatible material. Multifiber suture such as vicryl and silk can be soaked in the liquid ICG markers to create fluorescence. Monofilament suture can be dyed using the marker solutions listed. The addition of heat may help the NIR dye absorb into the suture. Biological suture such as chromatic suture may be used as the fluorescent marker binds to the tissue edge and maintains fluorescence.

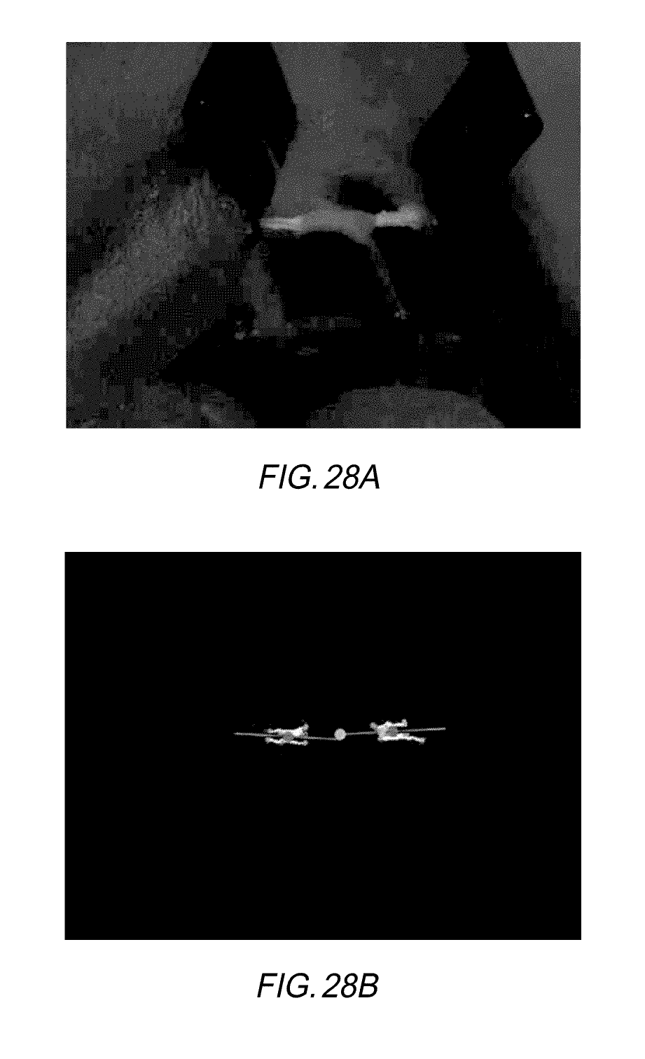

Understanding the location of sutures can be highly useful for manual and robotic procedures. Such knowledge can allow a surgeon to more easily keep track of a thread during a procedure. Additionally, robotic systems may benefit from thread tracking in difficult thread management applications. An automated segmentation routine could be used to determine suture position in an image as shown in FIG. 28A and FIG. 28B. After segmenting the suture in the image, as shown in FIG. 28B, the robot would be able to track the suture for the procedure. Also, tracking of stay sutures may obviate the need for other markers as features of interest, as the threads themselves could be tracked. By tracking the stay sutures, additional information from a surgical setup can be gleaned. For example, in a staged anastomosis, tracking of stay sutures quantifies the direction of tensioning during a procedure, useful for controlling tension through autonomous or semi-autonomous robotic intervention.

In another embodiment, the 2D information from a 2D NIR camera could be registered to a 3D camera and used to define and track markers in 3D. The 3D camera could be a plenoptic, structured light, stereo camera, lidar, or any other camera capable of imaging with depth information. This 3D information can then be used as part of an algorithm to aid the surgeon or robot during a procedure. For example, a robot could be commanded to approach a 3D marker with a tool or a surgeon could be informed about proper spacing and fixation of NIR sutures.

In another embodiment, positional information gleaned from the markers can be used to control the camera itself. This could be as simple as adjusting the intensity or gain of the image to maintain robust tracking changing the focus to maintain, a crisp image of the marker, or it could be more advanced. For example the camera may be mounted on a robotic arm and the marker position and orientation could in turn control the position and orientation of the camera. This may allow the marker to always be in a suitable field-of-view or angle relative to the camera. Additionally, the tracking of markers on tools could prevent tool-camera occlusions or collisions.

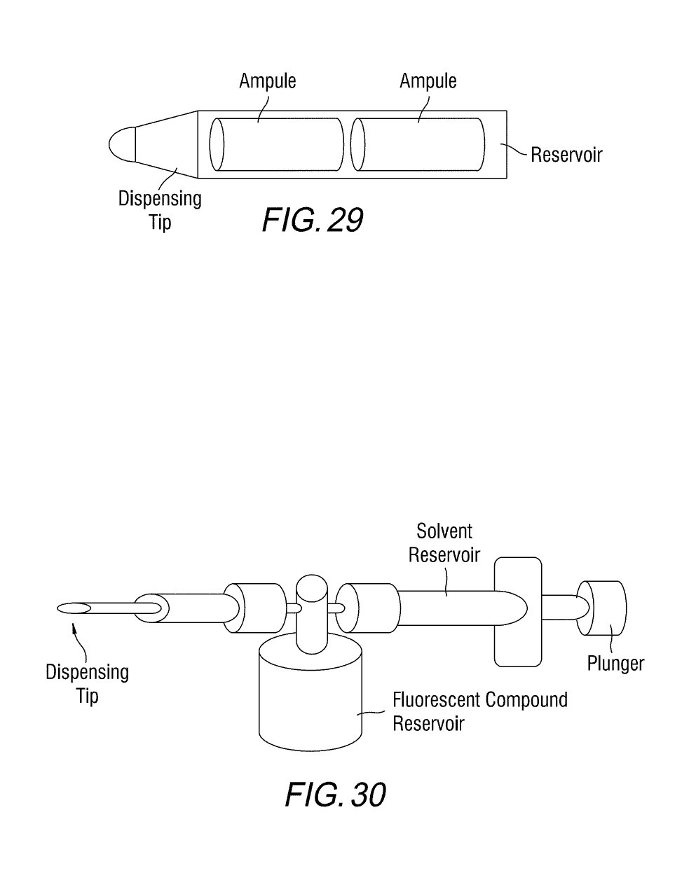

When applying liquid markers, it is desirable that the applied solution is uniform. Ideally, the liquid markers would be pre mixed and placed in sterile packaging. In an alternative embodiment, the individual components of the marker may be provided to the user as separate components. One advantage of this approach is an increase in the shelf-life of the liquid markers. In the exemplary embodiment, and as shown in FIG. 29, the components of the NIR marker may be packaged such that they are isolated from each other. Each ampoule may contain either a visualization agent, stabilization agent, or binding agent, with the third agent remaining in the reservoir. The ampules may be made from glass such that the agents encapsulated remain inert. The reservoir may be made from a compliant material that allows the users to apply force on the internal ampoules. When enough force has been applied, the ampules rupture, releasing their contents into the reservoir. The applicator may then be shaken to improve mixing of the agents. In another embodiment, a sphere may be inside the reservoir to improve mixing of the contents. In another embodiment, the dispensing tip contains a helical channel to aid in mixing the agents.

In another embodiment and as shown in FIG. 30, the agents may be combined with a mixing syringe. The mixing syringe may comprise a reservoir that contains one of the agents. Additional agents may be provided in sterile packaging that can be connected to the mixing syringe. When additional agents have been connected to the reservoir, they can be mixed by injection into the reservoir. Mixing can be accomplished by pumping the attached syringe. In another embodiment, a device such as a bone cement mixer can be used to homogenously combine the agents.

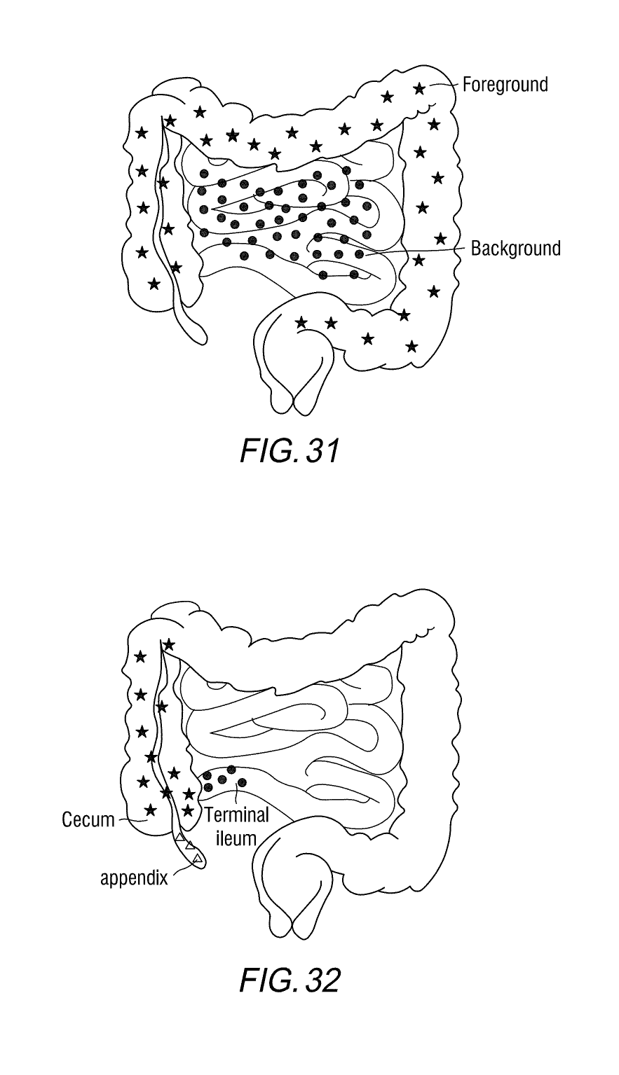

The marker composition, shape, and apparatus of the present disclosure have all been described in context of an open or laparoscopic surgical procedure. Herein, we discuss the potential applications of these markers in the surgical space. FIG. 31 illustrates a use of markers to distinguish between tissue types in the surgical scene. In one embodiment, markers are placed throughout the entire surgical field. The shape of the marker across the entire scene distinguishes tissue from foreground to background. Separating the surgical field into these two planes can be beneficial when performing a surgical task in one plane over the other, additional information is needed to visually reconstruct the surgical field, or information of the surgical environment is to be provided during an automated surgical task. In another embodiment, tissue types can be differentiated by placing a specific marker shape on similar tissues. This may be helpful for segmenting tissue types m standard laparoscopic images when the surgical scene is very uniform For instance, as shown in FIG. 32 markers may be used to differentiate tissue types or to mark regions of similar tissue. For example, markers can be used to identify the appendix, cecum, and terminal ileum even though all those regions appear similar to the untrained eye. A different marker or marker intensity could be used. This augments automatic segmentation methods and is useful for robotic and non-robotic procedures.

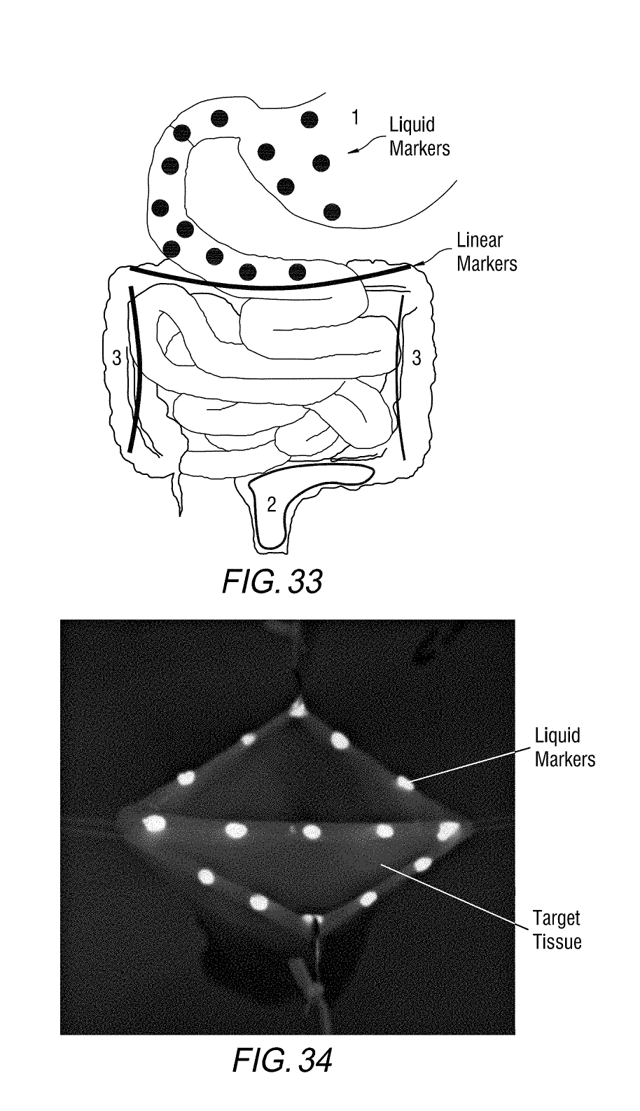

In another embodiment, markers may be placed in regions to designate one or more boundaries where a surgical tool should not be placed. As shown in FIG. 33, a marker can be used to designate a region of tissue where a surgeon should not place a surgical tool during a bowel procedure. The bounded regions may also define an area where a robotic surgical tool should not be placed during an automated task. Such bounded regions represent “no-fly zones” for the robotic system and help to enforce patient safety during an automated surgical task. Region one may be defined by a linear marker border and contain additional markers on the tissue. Region two may be defined by a completely enclosed linear marker border. Region three may be defined by a single linear marker. Each of the three regions may define a “no-fly zone”. In this way, the robotic motions can be restricted to the tissue of interest. The area defined as the “no-fly zone” can be defined by a space completely enclosed by a marker, a separated region with specific shaped markers inside it, or a separated region that has been designated as a “no-fly zone” by additional user interface.

In addition to identifying tissue types, markers can be placed in the surgical field to mark a tissue's landmarks and features. Knowing where specific features are on a tissue may make tissue motion easier to track in space. Additionally, marking features of a tissue such as a mesenteric edge may allow one to recreate tissue geometries in the surgical field. FIG. 34, for example, illustrates two ends of bowel tissue that have been tagged with a liquid, marker. The locations of the marker clearly identify the edges of each tissue in space. The markers can be used as target locations for an automated robotic platform to complete a surgical task. Additionally, consecutive markers can be interpolated to define the geometry of the target tissue, or generate a geometric path for an automated surgical system to follow.

By interpolating between markers, one can reconstruct the entire tissue edge and use this information to plan a surgical procedure. Additionally, reconstructing a tissue's geometry would be beneficial for automating a surgical task. In one example, all markers in FIG. 32 could be interpolated to highlight a tissue edge. This tissue edge could be visualized by an imaging system and tracked in 3-Dimensional space. The tissue edge could be further processed to form a path of which a robot could follow to carry out a surgical task, such as placing a suture for a bowel anastomosis. Alternatively, the markers may be used as targets for where a surgical task should be completed. One example of this strategy would include placing a marker on the mesenteric edge of tissue, and commanding a robotic grasping tool to pick up the mesentery tissue at the marker. In another embodiment, a laparoscope attached to a robotic positioner may be commanded to keep a marker in the camera's field of view. This would allow the laparoscope to be automatically positioned such that the target tissue is always visible to the surgeon.

In an additional embodiment, markers may be placed in the surgical scene for automatic positioning of a robotic platform “on the fly” during a surgical procedure by tracking tools or other instruments inside the body. Today, visual tracking of robotic tools is performed by visualization of optical markers placed on the back end of the tool. The liquid markers proposed here can be placed directly on the tool tip of the robotic instrument at any time during a procedure. Having the trackers directly in the surgical scene makes them more robust to surgical occlusion from equipment and personnel in the operating room. Additionally, a second marker can be placed on target tissue, and a closed loop positioning algorithm could be used to bring the robotic tool directly on top of the target tissue using a method known as visual servoing.

In an additional embodiment, combinations of these marker types and applications can be used to define a surgical procedure in the context of an autonomous or semi-autonomous robotic procedure. For example, markers may define a cut edge for a robotic resection algorithm. Or a series of markers may be used to define locations for a suturing robot. The added contrast and ability to discern between marker types can define more complex procedure plans using combinations of the embodiments described above, such as a “no-fly zone” combined with a cutting task, or a resection plan that updates based on interaction rules for different tissue types, or a suturing plan to connect two disparate tissues.

The specific embodiments described above have been shown by way of example in a surgical case and it should be understood that these embodiments may be susceptible to various modifications and alternative forms. It should be further understood that the claims are not intended to be limited to the particular forms disclosed, but rather to cover all modifications, equivalents, and alternatives falling within the spirit and scope of this. disclosure.

As used herein, the terms “comprises,” “comprising,” “including,” and “includes” are to be construed as being inclusive and open-ended. Specifically, when used in this document, the terms “comprises,” “comprising,” “including,” “includes,” and variations thereof, mean the specified features, steps or components included in the described features of the present disclosure. These terms are not to be interpreted to exclude the presence of other features, steps or components.

The following description includes features of the various embodiments.

(1) A system for tracking and control in medical procedures, the system comprising:
a device configured to deploy fluorescent material on at least one of an organ under surgery and a surgical tool;
a visual light source;
a fluorescent light source corresponding to an excitation wavelength of the fluorescent material;
an image acquisition and control element configured to control the visual light source and the fluorescent light source, and configured to capture and digitize at least one of resulting visual images and fluorescent images; and
an image-based tracking module configured to apply image processing to the visual and fluorescent images, the image processing detecting fluorescent markers on at least one of the organ and the surgical tool.

(2) The system of (1), further comprising:
a surgical robot; and
a visual servoing control module configured to receive tracking information from the image-based tracking module and to control the surgical robot, based on the tracking information, to perform a surgical operation.

(3) The system of (2), further comprising:
a manual control module configured to enable manual control of the surgical robot in place of control by the visual servoing control module.

(4) The system of (2), wherein the visual servoing control module is further configured to receive manual input and to control the surgical robot, based on the manual input, to perform a surgical operation.

(5) The system of (1), further comprising:
a surgical robot; and
a manual control module configured to receive manual input and execute master-slave control of the surgical robot.

(6) The system of (1), further comprising:
a display configured to display at least one of the visual images and the fluorescent images.

(7) The system of (1), wherein the image-based tracking module further identifies the organ or the surgical tool based on the detected fluorescent markers.

(8) The system of (1), wherein the image acquisition and control element further comprises:
a dynamic tunable filter configured to alternatively pass visual light and light emitted. by the fluorescent material, and
a charged coupled device configured to capture at least one of visual images and fluorescent images.

(9) The system of (6), wherein the display is stereoscopic or monoscopic

(10) The system of (1), wherein the image acquisition and control element generates stereoscopic or monoscopic images.

(11) The system of (6), wherein the stereoscopic display is further configured to display visual images and a color coded overlay of fluorescent images.

(12) The system of (6), wherein the stereoscopic display is further configured to display an augmented reality image by overlaying target points detected by the image-based tracking module.

(13) The system of (1), wherein the system is configured to provide at least one of visual, audio, and haptic feedback to a system operator, based on information provided by the image-based tracking module.

(14) The system of (1) wherein the system is configured to operate in each of a manual mode, a semi-autonomous mode, and an autonomous mode.

(15) The system of (1), wherein image-based tracking module identifies virtual boundaries based on the detected fluorescent markers to designate critical structures.

(16) The system of (15). further comprising:
a detection device configured to determine whether a surgical tool has passed a boundary and to provide constraints on motion or provide alarms when the boundary has been crossed in order to protect the critical structures.

(17) The system of (1). wherein the fluorescent light source is a near-infrared (NIR) light source.

(18) The system of (1), wherein the device that deploys the fluorescent material is configured to deploy the fluorescent material by spraying, painting, attachment, tissue injection, or intravenous injection.

(19) A method for performing a medical procedure, the method comprising the steps of:
deploying fluorescein material on at least one of an organ under surgery and a surgical tool;
illuminating the organ, the surgical tool, or both, with a visual light source and a fluorescent light source, the fluorescent light source corresponding to an excitation wavelength of the fluorescent material;
capturing and digitizing images resulting from the illumination by the visual light source and the fluorescent light source; and
applying image processing to the digitized images, the image processing detecting fluorescent markers on at least one of the organ and the surgical tool.

(20) The method according to (19), further comprising:
generating tracking information by tracking the organ, the surgical tool, or both based on the detected fluorescent markers.

(21) The method of (19), further comprising:
controlling a surgical robot, based on the tracking information, to perform a surgical operation.

(22) The method of (21), further comprising:
receiving manual input; and controlling the surgical robot, based on the manual input, to perform the surgical operation.

(23) The method of (19), further comprising:
receiving manual input; and
executing master-slave control of a surgical robot based on the on manual input.

(24) The method of (19), further comprising:
providing a stereoscopic or monoscopic display of the digitized images.

(25) The method of (19), wherein the step of capturing and digitizing images further
comprises generating stereoscopic or monoscopic images.

(26) The method of (24), further comprising:
displaying visual images and a color coded overlay of fluorescent images.

(27) The method of (24), further comprising: displaying an augmented reality image by overlaying target points detected by the image-based tracking module.

(28) The method of (19), further comprising:
providing at least one of visual, audio, or haptic feedback to a system operator, based on the tracking information.

(29) The method of (19), further comprising:
identifying the organ or the surgical tool based on the detected fluorescent markers.

(30) The method of (19), further comprising:
performing a surgical procedure based on the detected fluorescent markers.

(31) The method of (19), further comprising:
designating critical structures by identifying virtual boundaries based on the detected fluorescent markers.

(32) The method of (31), further comprising:
determining whether a surgical tool has passed a boundary and providing constraints on motion or providing alarms when the boundary has been crossed in order to protect the critical structures.

(33) A system for tracking and control in medical procedures, the system comprising:
means for deploying fluorescent material on at least one of an organ under surgery and a surgical tool;
a visual light source;
a fluorescent light source corresponding to an excitation wavelength of the fluorescent material;

means for controlling the visual light source and the fluorescent light source;
means for capturing and digitizing at least one of resulting visual images and fluorescent images; and
means for applying image processing to the visual and fluorescent images, the image processing detecting fluorescent markers on at least one of the organ and the surgical tool.

(34) A system for tracking and control in medical procedures, the system comprising:
a visual light source;
one or more fluorescent markers;
a fluorescent light source corresponding to an excitation wavelength of the fluorescent markers; and
circuitry configured to
control the visual light source and the fluorescent light source,
track a position of the fluorescent markers within visual and fluorescent images by real-time tracking.

While certain embodiments have been described, these embodiments have been presented by way of example only, and are not intended to limit the scope of the inventions. Indeed the novel methods and systems described herein may be embodied in a variety of other forms; furthermore, various omissions, substitutions, and changes in the form of the methods and systems described herein may be made without departing from the spirit of the inventions. As used herein the words “a” and “an” and the like carry the meaning of “one or more.” The accompanying claims and their equivalents are intended to cover such forms or modifications as would fall within the scope and spirit of the inventions.

1. A system for tracking and control in medical procedures, the system comprising:
processing circuitry configured to
control a visual light source and a fluorescent light source,
capture and digitize resulting visual images and fluorescent images of an organ under surgery having one or more fluorescent markers thereon,
apply image processing to the digitized visual images and fluorescent images, the image processing detecting at least one fluorescent marker of the one or more fluorescent markers on the organ, and
track a three-dimensional position of the detected at least one fluorescent marker within the visual images and fluorescent images based upon a fluorescence of the detected at least one fluorescent marker on the organ under surgery.

2. The system of claim 1,
wherein the processing circuitry is further configured to
obtain tracking information, and
control a surgical robot, based on the tracking information, to perform an automated surgical operation.

3. The system of claim 2, wherein the processing circuitry is further configured to enable manual control of the surgical robot in place of the automated surgical operation.

4. The system of claim 2, wherein the processing circuitry is further configured to control display of at least one of the visual images and the fluorescent images on a display.

5. The system of claim 1, wherein the processing circuitry is further configured to identify the organ under surgery based on the detected at least one fluorescent marker.

6. The system of claim 4, wherein the processing circuitry is further configured to control display of visual images and a color coded overlay of fluorescent images.

7. The system of claim 4, wherein the processing circuitry is further configured to control display of an augmented reality image by overlaying detected target points.

8. The system of claim 1, wherein the processing circuitry is further configured to provide at least one of visual, audio, and haptic feedback to a system operator.

9. The system of claim 1, wherein application of the one or more fluorescent markers to the organ under surgery includes spraying the one or more fluorescent markers.

10. A method for performing a medical procedure, the method comprising:
illuminating, via processing circuitry, an organ under surgery with a visual light source and a fluorescent light source, the fluorescent light source corresponding to an excitation wavelength of one or more fluorescent markers applied to the organ under surgery;
capturing and digitizing, by the process circuitry, resulting visual images and fluorescent images from the illumination by the visual light source and the fluorescent light source of the organ under surgery having one or more fluorescent markers applied thereon;
applying, by the processing circuitry, image processing to the digitized visual images and fluorescent images, the image processing detecting at least one fluorescent marker of the one or more fluorescent markers on the organ under surgery; and
tracking, by the processing circuitry, a three-dimensional position of the at least one fluorescent marker within the visual images and fluorescent images based on a fluorescence of the detected at least one fluorescent marker on the organ under surgery.

11. The method 10, further comprising:
generating, by the processing circuitry, a dynamic virtual boundary based on the detected at least one fluorescent marker on the organ under surgery within the visual images and fluorescent images such that a position of the dynamic virtual boundary is maintained in correspondence with a position of the organ under surgery within a frame of the visual images and fluorescent images;
determining, by the processing circuitry, whether a surgical tool has passed the dynamic virtual boundary based on a tracked position of a respective at least one fluorescent marker on the surgical tool; and
generating, by the processing circuitry, an alert when the dynamic virtual boundary has been crossed.

12. The method of claim 11, further comprising:
constraining, by the processing circuitry, motion of the surgical tool when the dynamic virtual boundary has been crossed.

13. The method of claim 10, further comprising:
applying the one or more fluorescent markers to the organ under surgery; and
combining the one or more fluorescent markers with an adhesive prior to the applying the one or more fluorescent markers to the organ under surgery, wherein the adhesive is a cyanoacrylate.

14. The method of claim 10, further comprising:
applying the one or more fluorescent markers to the organ under surgery,
wherein the applying the one or more fluorescent markers to the organ under surgery includes mechanically-securing the one or more fluorescent markers to the organ under surgery via a fastener.

15. The method of claim 10, further comprising:
tracking, by the processing circuitry, the three-dimensional position of the at least one fluorescent marker within the visual images and the fluorescent images based upon a checkered pattern of a predefined shape, the checkered pattern being fabricated with the one or more fluorescent markers.

16. The method of claim 11, wherein the at least one fluorescent marker on the surgical tool is a checkered pattern of a predefined shape.

17. The method of claim 10, wherein the one or more fluorescent markers are deployed on the organ under surgery by a marking tool including a solution reservoir, a dispensing needle, and second processing circuitry configured to control a pump.

18. The method of claim 10,
applying the one or more fluorescent markers to the organ under surgery,
wherein the one or more fluorescent markers are applied to the organ under surgery by application of compressive force to a reservoir of an applicator, the applicator including the reservoir, a breakable ampoule housing the one or more fluorescent markers, and a dispensing tip.

19. A system for tracking and control in medical procedures, the system comprising:
a visual light source;
one or more fluorescent markers applied to an organ under surgery;
a fluorescent light source corresponding to an excitation wavelength of the one or more fluorescent markers;
processing circuitry configured to
control the visual light source and the fluorescent light source,
capture and digitize resulting visual images and fluorescent images of the organ under surgery having the one or more fluorescent markers thereon,
apply image processing to the digitized visual images and fluorescent images, the image processing detecting at least one fluorescent marker of the one or more fluorescent markers on the organ under surgery, and
track a three-dimensional position of the detected at least one fluorescent marker within the visual images and fluorescent images based upon a fluorescence of the detected at least one fluorescent marker on the organ under surgery.

20. The system of claim 19, wherein the processing circuitry is further configured to
generate a dynamic virtual boundary based on the detected at least one fluorescent marker on the organ under surgery within the visual images and fluorescent images such that a position of the dynamic virtual boundary is maintained in correspondence with a position of the organ under surgery within a frame of the visual images and fluorescent images,
determine whether a surgical tool has passed the dynamic virtual boundary based on a tracked position of a respective at least one fluorescent marker on the surgical tool, and
generate an alert when the dynamic virtual boundary has been crossed.
